(19) United States
(12) Patent Application Publication
(10) Pub. No.: US 2017/0173578 A1
(43) Pub. Date: Jun. 22, 2017

(54) MICROFLUIDIC DEVICES FOR THE RAPID DETECTION OF ANALYTES

(71) Applicant: BOARD OF REGENTS, THE UNIVERSITY OF TEXAS SYSTEM, Austin, TX (US)

(72) Inventors: Richard M. Crooks, College Station, TX (US); Christopher Renault, Austin, TX (US); Stephen E. Fosdick, Austin, TX (US); Xiang Li, Austin, TX (US)

(21) Appl. No.: 15/126,030

(22) PCT Filed: Mar. 13, 2015
(86) PCT No.: PCT/US15/20569

§ 371 Date: Sep. 14, 2016

(51) Int. Cl. B01L 3/00; G01N 27/30; G01N 27/48; G01N 27/403; G01N 33/66; G01N 33/68

(52) U.S. Cl. CPC B01L 3/5027; G01N 33/66; G01N 33/68; B01L 3/502746; G01N 27/48; G01N 27/403; G01N 27/308; B01L 2300/126; B01L 2300/0816; B01L 2300/161; B01L 2300/165; B01L 2400/086; B01L 2300/0627; B01L 2300/0645; B01L 2300/0887

(57) ABSTRACT

Provided herein are paper-based microfluidic devices that can be configured to induce fast fluid flow through a hollow microfluidic channel under low applied pressure. The microfluidic devices can comprise a fluid inlet, a fluid outlet, and a hollow channel fluidly connecting the fluid inlet and the fluid outlet, so as to form a fluid flow path from the fluid inlet to the fluid outlet. The hollow channel can comprise a fluid flow path defined by a floor, two or more side walls, and optionally a ceiling. One or more of the interior surfaces of the hollow channel can comprise a hydrophilic material. The hydrophilic material can drive fluid flow through the hollow channel, allowing for fast fluid flow through the hollow microfluidic channel under low applied pressure. The devices are well suited for use in numerous sensing applications, for example, quantitative, low limit-of-detection, and/or point-of-care paper analytical devices.

### CROSS-REFERENCE TO RELATED APPLICATIONS

This application claims benefit of U.S. Provisional Application No. 61/953,469, filed Mar. 14, 2014, which is hereby incorporated herein by reference in its entirety.

### STATEMENT REGARDING FEDERALLY SPONSORED RESEARCH OR DEVELOPMENT

This invention was made with government support under Contract No. HDTRA-1-13-1-0031 awarded by the Department of Defense/Defense Threat Reduction Agency (DTRA). The government has certain rights in this invention.

### FIELD OF THE DISCLOSURE

This invention relates generally to microfluidic devices for the rapid detection of analytes in a fluid.

### BACKGROUND

Point-of-care (POC) diagnostics are inherently attractive in many resource-limited settings where the healthcare, transportation, and distribution infrastructure is underdeveloped and underfunded. The main advantage of a POC diagnostic is the ability to diagnose disease without the support of a laboratory infrastructure; this increases access, removes the need for sample transport, and shortens turnaround times from weeks (or months) to hours. As a result, more patients are effectively diagnosed, enabling faster and more complete treatment. Although commercial paper-based sensors have been around for about 25 years (e.g., pregnancy test and glucose test strips), few paper POC devices have been successfully commercialized. Such failure to produce trustworthy paper POC devices is a combination of many factors, including poor limits of detection (LOD), high non-specific adsorption (NSA), unstable reagents, long analysis time, complex user-technology interface, detection method, and poor sensitivity. There is a need for paper point-of-care (POC) devices that are cheap, user friendly, robust, sensitive, and portable. Such devices pose an effective solution to the existing economic and healthcare accessibility problems in underdeveloped countries, as well as the growing trend in more affluent societies to become better informed in terms of its health.

Most paper-based analytical devices rely on capillary flow to control both direction and rate of fluid flow though the device. While capillary driven-flow is advantageous in many regards, the presence of the cellulose matrix introduces several difficulties such as low rates of convective mass transfer, significant non-specific adsorption due to the high surface area of the cellulose fibers, and a size restriction on the mobility of objects within cellulose matrix due to the size-exclusion properties of paper. Microfluidic devices containing hollow channels provide a fluid flow path that is unencumbered by a cellulose matrix. However, without a cellulose matrix defining a fluid flow path, there is no driving force for fluid flow through the hollow hydrophobic channel. In such cases, pressure must be applied externally to drive fluid flow through the hollow hydrophobic channel (e.g., using a syringe pump). As a consequence, existing hollow channel microfluidic devices are not well suited for many POC applications.

It is an object of this invention to provide microfluidic devices that can induce fast fluid flow through a channel under low applied pressure.

It is also an object of this invention, to provide paper-base microfluidic devices that have a quantitative read out, low limits of detection, and low cost of instrumentation.

### SUMMARY OF THE DISCLOSURE

Provided herein are microfluidic devices that can be configured to induce fast fluid flow through a hollow microfluidic channel under low applied pressure. The microfluidic devices can comprise a fluid inlet, a fluid outlet, and a hollow channel fluidly connecting the fluid inlet and the fluid outlet, so as to form a fluid flow path from the fluid inlet to the fluid outlet.

The hollow channel can comprise a fluid flow path defined by a floor, two or more side walls, and optionally a ceiling. Together, the floor, the two or more side walls, and the ceiling, when present, define a conduit or void space through which fluid (e.g., an aqueous solution) can flow during device operation. One or more of the interior surfaces of the hollow channel (e.g., the floor, a side walls, the ceiling, or a combination thereof) can comprise a hydrophilic material. The hydrophilic material can be porous hydrophilic material, such as paper. The hydrophilic material can drive fluid flow through the hollow channel, allowing for fast fluid flow through the hollow microfluidic channel under low applied pressure.

The microfluidic devices can be used in analytical applications, for example, to assay a fluid sample for the presence of one or more analytes. In some cases, the microfluidic device can further include an assay reagent that facilitates the detection, identification, and/or quantification of an analyte present in the fluid sample. In some cases, the microfluidic device can further include a detection device, for example, an image scanner, a camera, a fluorometer, a spectrometer, or an electroanalytical device which can be used to detect and/or measure the analyte, the assay reagent, a substance indicative of the analyte, or a combination thereof. In certain embodiments, the microfluidic device can include one or more electrodes in electrochemical contact with the hollow channel that can be used to detect and/or measure the analyte, the assay reagent, a substance indicative of the analyte using conventional electroanalytical methods. In some embodiments, the electrode can be a bulk conductive electrode.

The devices described herein can be inexpensive, user friendly (they employ electrochemical detection without any washing steps), sensitive, portable, robust, efficient, rapid (completion of analysis in minutes), and can detect low concentrations of analytes in a fluid sample. The microfluidic devices can exhibit electrochemical and hydrodynamic behavior similar to traditional glass and plastic microfluidic electrochemical devices. As such, the devices are well suited for use in numerous sensing applications, for example, quantitative, low limit-of-detection, and/or point-of-care paper analytical devices.

### BRIEF DESCRIPTION OF THE DRAWINGS

FIGS. 1A-1D are drawings illustrating example microfluidic devices.

FIGS. 2A-2D are illustrations of individual printed layers used to form example microfluidic devices. After printing and curing, regions were removed to form hollow channels.

FIGS. 3A-3C are micrographs of paper-based hollow channel devices. The devices contain a 1 mm-wide wax barrier (FIG. 3A), a 180 μm-thick paper barrier (FIG. 3B), and a 1 cm-long paper barrier (FIG. 3C). The positions of the barriers are indicated by the arrows superimposed on the micrographs.

FIGS. 4A-4D display a photograph of a microfluidic paper analytical device (PAD) and three graphs showing the time for fluid to flow through the device. FIG. 4A is a photograph of the unfolded PAD used to measure flow rates. FIG. 4B is a graph showing the distance flowed by an aqueous 5.0 mM erioglaucine solution in a hollow channel (hollow circles) and a paper channel (filled circles) as a function of time. The error bars represent the standard deviation of four independent experiments. The hollow and paper channels were both ˜180 μm tall by 2.5 mm wide. The pressure at the inlet was 1.2 mbar. FIG. 4C is a graph showing the flow rate calculated from the derivative of the data in FIG. 4B. FIG. 4D is a graph showing the same parameters as in 4B, but with the indicated pressures at the inlet. The two superimposed curves for the paper channels were obtained at 1.2 and 2.7 mbar.

FIGS. 5A-5D display a PAD used for glucose and bovine serum albumin (BSA) assays and two graphs showing the calibration curve of analyte concentrations with color intensity. FIG. 5A shows the PAD used for the glucose and BSA assays. FIG. 5B is a photograph of the results of the glucose and BSA PAD assay performed. The picture was taken 5 min after introducing 80 μL of sample containing 75 μM BSA and 20 mM glucose in PBS 1× buffer (12 mM phosphate buffer, pH 7.4, 137 mM NaCl, and 2.7 mM KCl). The color of the wells containing the glucose assay reagents turned from white to brown in presence of glucose. The color of the wells containing the BSA assay reagents turned from yellow to blue in presence of BSA. For intermediary concentrations of BSA the mixture of yellow and blue colors gives a green coloration to the wells. FIGS. 5C and 5D are calibration curves for the glucose (Figure C) and BSA (Figure D) assays, respectively. The data were measured with two different devices, each one giving five replicates.

FIGS. 6A-6G are optical micrographs of three independent hollow-channel paper analytical devices (HC-PADs) and an illustration of the cross-section of a HC-PAD. FIGS. 6A, 6C, and 6E are optical micrographs of the HC-PADs in the absence of water and FIGS. 6B, 6D, and 6F are in presence of water. The dashed lines indicate the position of the HC. The “wet” micrographs are taken 5 min after adding water in the HC-PADs. FIG. 6G is a schematic of the cross-section of a HC-PAD.

FIGS. 7A and 7B are graphs showing results from chronoamperometry (CA) for HC-PADs. FIG. 7A shows the change in current over time corresponding to a potential step from −0.100 V vs Ag/AgCl to +0.400 V vs Ag/AgCl. Ferrocene methanol concentration, [FcMeOH]=250 μM (in PBS 1×); screen-printed working electrode (SWE) area=0.040±0.004 cm². The CAs were recorded using 3 independent devices. The ohmic resistance was not compensated. FIG. 7B shows the change in current for device 1 as a function of t^{-1/2}. The inset shows the periodic noise observed at long time scales. The design of the HC-PADs used to record the CAs is shown in FIG. 13A.

FIG. 8 is a scatter plot showing the collection efficiencies obtained for three independent HC-PADs (each shape corresponds to a different device).

FIGS. 9A-9B are graphs showing the results from CA and the average linear flow rate as a function of pressure for a HC-PAD. FIG. 9A illustrates the results from CA and shows the change in current as a function of time corresponding to the reduction of FcMeOH⁺ at the collector electrode in a cellulose-filled channel. The design of the PAD used to record the CAs is similar to the one shown in FIG. 16A except that cellulose is left in the channel. The pressures within the channel were 2.6, 4.1, and 5.6 mbar for the black, red and blue lines, respectively. Each time the pressure was modified, the solution was allowed to flow for at least 10 min to equilibrate the flow within the channel. The potential of the generator was maintained at 0.600 V vs standard calomel electrode (SCE) for 15 min and then stepped to −0.200 V vs SCE at t=0 min. The potential of the collector was kept at −0.200 V vs SCE during the entire experiment. S_{gen}=S_{col}=0.2 cm×0.2 cm (0.04 cm²); [FcMeOH]=250 μM (in pH 7.4 PBS 1×). FIG. 9B shows the average linear flow rate as a function of the pressure. The error bars correspond to the uncertainty of l_{G-C} (0.2 mm) and t_{G-C} (0.1 min). The red line is a linear fit of the experimental data (slope=0.0056±0.0002 mm/(s mbar); R²=0.995).

FIGS. 10A-10C illustrates a HC-PAD and two graphs showing the variation in volume and volumetric flow rate in the HC-PAD. FIG. 10A shows the experimental setup used to measure the volumetric flow rate. The blue color represents the plastic holder and the orange arrows indicate the direction of flow. The HC-PADs used to measure volumetric flow rate Q are similar to the ones used to measure average linear flow rate uₐᵥ. This means that three carbon electrodes are screen-printed on the ceiling of the HC. However, in that particular experiment, the electrodes are not connected to a potentiostat. FIG. 10B shows the variation of volume in the outlet reservoir measured using the experimental setup shown in FIG. 10A. The black, red, and blue colors correspond to pressures of 2.1, 2.9, and 4.1 mbar, respectively. The lines are least-squares fits of the data. The errors bars represent the standard deviation obtained for three replicate measurements. FIG. 10C shows the volumetric flow rate plotted as a function of pressure. The three colors correspond to three independently fabricated devices. The error bars correspond to the standard deviation of three replicate measurements per device.

FIG. 11 shows the geometry used for simulations of hydrodynamics and electrochemistry with convection. Boundaries are outlined based on their physical representation.

FIG. 12 shows the geometry used for simulations of electrochemistry in absence of convection. Boundaries are outlined based on their physical representation.

FIGS. 13A-13D show an illustration of a HC-PAD and three graphs showing the recording of the CV, variation of the peak current potential, and the peak currents for the HC-PAD. FIG. 13A is a 3D schematic illustration of a three-electrode HC paper electrochemical cell. The scheme is not drawn to scale. FIG. 13B is the CV recorded using a HC-PAD like the one presented in FIG. 13A. The channel was filled with a solution containing 250μM FcMeOH and PBS 1×. The black, red, blue, and green lines correspond to v=10, 20, 50 and 100 mV/s, respectively. The solution was not flowing during the experiments. R_{comp}=7 kΩ and the geometric area of the SWE was 0.040±0.004 cm². FIG. 13C shows the variation of the anodic (red) and cathodic (black) peak-current potentials with v. FIG. 13D shows the anodic (red) and cathodic (black) peak currents as a function of v^{1/2}. In FIGS. 13C and 13D, the error bars represent the standard deviations observed using three independent devices.

FIGS. 14A-14C are graphs depicting the change in current per time and a concentration profile of the analyte for a HC-PAD. FIG. 14A shows the experimentally determined CA corresponding to the oxidation of FcMeOH in the HC-PAD shown in FIG. 13A (black line). The potential was stepped from −0.100 V to +0.400 V vs Ag/AgCl, [FcMeOH]=250μμM (in PBS 1×), and SWE area=0.040±0.004 cm². In that particular experiment the ohmic resistance was not compensated. The blue line is a plot of the Cottrell equation. FIG. 14B is a numerical simulation of the experiment represented in FIG. 14A (red line). The blue line is an extrapolation of the linear path (2 to 7 s) of the simulated CA. FIG. 14C shows the concentration profiles of FcMeOH derived from the simulated CAs at 5, 15, and 45 s. The position of the WE in the channel is indicated by the thick black line. The white dots represent the cellulose fibers in the paper floor.

FIG. 15 are cross-sectional optical micrographs of a hollow channel. These micrographs were taken under white (a), blue (b), and UV illumination (c). Micrographs (d) and (e) are close-ups of the top layer. These micrographs were taken under white and blue light, respectively.

FIG. 16 are optical micrographs of three independent HC-PADs (a,b; c,d; e,f) in absence and in presence of water in the HC. The orange dashed lines indicate the position of the HC. The “wet” micrographs are taken 5 min after adding water in the HC-PADs.

FIGS. 17A-17B are results from CA using the design of the HC-PADs FIG. 12A. FIG. 17A is a CAs corresponding to a potential step from −0.100 V vs Ag/AgCl to +0.400 V vs Ag/AgCl. [FcMeOH]=250 μM (in PBS 1×); SWE area=0.040±0.004 cm². The CAs were recorded using 3 independent devices. The ohmic resistance was not compensated. FIG. 17B is a CA of Device 1 (shown in FIG. 17 (top) plotted as a function of t^{-1/2}. The inset shows the periodic noise observed at long time scales.

FIGS. 18A-18B are schematic illustrations and photographs of a HC-PAD. FIG. 18A is a schematic illustration demonstrating qualitatively the laminar flow in a HC-PADs. Two aqueous solutions containing 1.0 mM tartrazine (yellow) or 50.0 mM resazurin (blue) were introduced at the two inlets. FIG. 18B is a schematic illustration of the HC-PAD used to quantify laminar flow. The two WEs partially cross the HCs. The CE and RE were, respectively, a Pt wire and a glass Ag/AgCl, 1 M KCl electrode placed in the outlet reservoir. The red and blue reservoirs were filled with a saline solution (0.5 M NaCl) containing 1.0 mM Fe(phen)SO₄ and 1.0 mM FcDM, respectively. The solutions were allowed to flow for 2 min, and then the flow was stopped immediately prior to recording the CVs. v=100 mV/s; R_{comp}=6 kΩ; SWE1=0.029 cm²; SWE2=0.021 cm².

FIG. 19 is collection efficiencies obtained for three independent HC-PADs (each color corresponds to a different device).

FIG. 20A-20B are results from CA using a HC-PAD. FIG. 20A a CA corresponding to the reduction of FcMeOH+ at the collector electrode in a cellulose-filled channel. The design of the PAD used to record the CAs is similar to the one shown in FIG. 20A except that cellulose is left in the channel. The pressures within the channel were 2.6, 4.1, and 5.6 mbar for the black, red and blue lines, respectively. Each time the pressure was modified, the solution was allowed to flow for at least 10 min to equilibrate the flow within the channel. The potential of the generator was maintained at 0.600 V vs SCE for 15 min and then stepped to −0.200 V vs SCE at t=0 min. The potential of the collector was kept at −0.200 V vs SCE during the entire experiment. Electrode surface area S_{gen}=S_{col}=0.2 cm×0.2 cm (0.04 cm²); [FcMeOH]=250 μM (in pH 7.4 PBS 1×). (b) Average linear flow rate (uₐᵥ) as a function of the pressure. The values of uₐᵥ were calculated using eq 2 (γ=1; l_{G-C}=2.5 mm) and the time delays t_{G-C} shown in FIG. 20A. The error bars correspond to the uncertainty of l_{G-C} (0.2 mm) and t_{G-C} (0.1 min). The red line is a linear fit of the experimental data (slope=0.0056±0.0002 mm/(s mbar); R²=0.995).

FIGS. 21A-21C illustrates a HC-PAD design and analysis if the volumetric flow rate. FIG. 21A is the experimental setup used to measure the volumetric flow rate. The blue color represents the plastic holder and the other colors have the same representations as in FIG. 12A. The orange arrows indicates the direction of flow. The HC-PADs used to measure Q are similar to the ones used to measure uₐᵥ. This means that three carbon electrodes are screen-printed on the ceiling of the HC. However, in that particular experiment, the electrodes are not connected to a potentiostat. FIG. 21B is a graph showing the typical variation of volume in the outlet reservoir measured using the experimental setup shown in FIG. 21A. The black, red, and blue colors correspond to pressures of 2.1, 2.9, and 4.1 mbar, respectively. The lines are least-squares fits of the data. The errors bars represent the standard deviation obtained for three replicate measurements. FIG. 21C is a graph showing the volumetric flow rate plotted as a function of pressure. The three colors correspond to three independently fabricated devices. The error bars correspond to the standard deviation of three replicate measurements per device.

FIGS. 22A-22C illustrates a HC-PAD and two graphs showing the currents and the average linear flow in the HC-PAD. FIG. 22A is a schematic illustration showing the HC electrochemical PAD used in the experiments. The description is the same as in FIG. 13A. The scheme is not drawn to scale. FIG. 22B shows the currents measured at the generator (black line) and collector (red line) electrodes during the CA experiment. The potential was stepped from −0.200 V vs SCE to 0.600 V vs SCE; S_{gen}=S_{col}=0.2 cm×0.2 cm (0.04 cm²); [FcMeOH]=250 μM (in PBS 1×). The CE and RE were, respectively, a Pt wire and an SCE placed in the outlet reservoir. Each time the pressure was modified, the solution was allowed to flow for at least 5 min to ensure limiting behavior. The red arrow indicates the time delay, t_{G-C}, between the generation and the collection of FcMeOH⁺. FIG. 22C shows the average linear flow rate (uₐᵥ) measured for the generation-collection experiment. The green, red, and blue circles correspond to measurements obtained using three independent devices. The error bars correspond to the standard deviation of at least 10 replicate measurements of t_{G-C}. The black line is a least-squares fit to the data. (slope=2.7±0.2 mm/(s mbar); R²=0.956).

FIGS. 23A-23C illustrates two CV recordings and a plot of current for a HC-PAD. FIG. 23A is CV recordings of 250 μM FcMeOH in PBS 1× as a function of scan rate. The black, red, blue, and green lines correspond to v=5, 50, 100, and 500 mV/s, respectively. The pressure drop within the HC was held constant at 0.3 mbar. R_{comp}=7 kΩ; SWE area=0.040±0.004 cm². FIG. 23B is CV recordings of 250 μM FcMeOH in PBS 1× as a function of the pressure in the HC. The black, red, blue, and green lines correspond to P=0, 0.3, 1.5, and 2.9 mbar, respectively. v=50 mV/s; R_{comp}=7 kΩ; SWE area=0.040±0.004 cm². FIG. 23C is a plot of the experimental i_{L} plotted as a function of (uₐᵥ)^{1/3} (black triangles). The values of uₐᵥ, were calculated using the experimentally determined value of P and the slope of the linear fit in FIG. 22C. The vertical and horizontal error bars correspond to the standard deviation obtained using three independent devices and the standard deviation of the fit in FIG. 22C, respectively. The blue line is a least-squares fit of the experimental data (slope=−1.63±0.02 μA/(mm/s)^{1/3}; R²=0.999). Values of i_{L} obtained by numerical simulation are plotted as red triangles. The HC-PADs similar to the one presented in FIG. 13A; that is, with the WE (carbon paste), CE (carbon paste) and RE (Ag/AgCl paste) positioned in the HC, were used to carry out the experiments shown in FIG. 17.

FIG. 24 shows the geometry of the unit cell used to describe the porosity of the paper floor. The white and gray colors represent the cellulose fibers and the pores, respectively.

FIG. 25 shows the top view of Whatman grade 1 chromatography paper obtained by scanning electron microscopy.

FIG. 26 shows a 3D view of an exemplary HC-PAD before assembly (folding).

### DETAILED DESCRIPTION OF THE DISCLOSURE

The devices and methods described herein may be understood more readily by reference to the following detailed description of specific aspects of the disclosed subject matter, figures and the examples included therein.

Before the present devices and methods are disclosed and described, it is to be understood that the aspects described below are not intended to be limiting in scope by the specific devices and methods described herein, which are intended as illustrations. Various modifications of the devices and methods in addition to those shown and described herein are intended to fall within the scope of that described herein. Further, while only certain representative devices and method steps disclosed herein are specifically described, other combinations of the devices and method steps also are intended to fall within the scope of that described herein, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

“Multiple” or “plurality” as used herein, is defined as two or more than two.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Devices

Provided herein are microfluidic devices configured to induce fast fluid flow through a hollow microfluidic channel under low applied pressure. The microfluidic devices can comprise a fluid inlet, a fluid outlet, and a hollow channel fluidly connecting the fluid inlet and the fluid outlet, so as to form a fluid flow path from the fluid inlet to the fluid outlet.

The hollow channel can comprise a fluid flow path defined by a floor, two or more side walls, and optionally a ceiling. Together, the floor, the two or more side walls, and the ceiling, when present, define a conduit or void space through which fluid (e.g., an aqueous solution) can flow during device operation. The interior of the hollow channel through which fluid flows contains one or more regions along the length of the fluid flow path that are substantially free of a matrix material (e.g., that are substantially free of a porous solid such as paper or cellulose through which fluid flows during device operation). In some embodiments, the entire length of the hollow channel can be substantially free of matrix material.

The floor, the two or more side walls, and/or optionally the ceiling can be substantially impermeable to fluid flow, so as to form boundaries that define a path for fluid flow through the hollow channel. For example, the floor, the two or more side walls, and/or optionally the ceiling can be substantially hydrophobic, so as to form boundaries that restrict fluid flow within the hollow channel, thereby defining a path for the flow of an aqueous solution through the hollow channel.

One or more of the interior surfaces of the hollow channel (e.g., the floor, a side walls, the ceiling, or a combination thereof) can comprise a hydrophilic material. The hydrophilic material can comprise a portion of one or more of the interior surfaces of the hollow channel (e.g., a portion of the floor, a portion of a side wall, a portion of the ceiling, or a combination thereof). Alternatively, the hydrophilic material can comprise the entirety of one or more of the interior surfaces of the hollow channel (e.g., the entirety of the floor, the entirety of a side wall, the entirety of the ceiling, or a combination thereof). The hydrophilic material can be porous. The hydrophilic material can drive fluid flow through the hollow channel, allowing for fast fluid flow through the hollow microfluidic channel under low applied pressure.

The dimensions of the hollow channel can be varied so as to provide a device having performance characteristics desired for a particular application. The hollow channel can be fabricated so as to have a variety of cross-sectional dimensions. For example, in some embodiments, the hollow channels can have a substantially square or rectangular cross-sectional shape. Though referred to herein as “microfluidic devices,” the devices described herein can include hollow channels having dimensions (e.g., width and/or height) on the micron or millimeter scale.

The hollow channel can have any suitable width, provided that the channel width is compatible with device function. In some embodiments, the hollow channel can have a width (e.g., defined as the distance between two side walls of the hollow channel) of about 50 mm or less (e.g., about 45 mm or less, about 40 mm or less, about 35 mm or less, about 30 mm or less, about 25 mm or less, about 20 mm or less, about 15 mm or less, about 10 mm or less, about 7.5 mm or less, about 5 mm or less, about 2.5 mm or less, about 2 mm or less, about 1.5 mm or less, about 1 mm or less, or about 0.5 mm or less). In some embodiments, the hollow channel can have a width of at least about 0.1 mm (e.g., at least about 0.5 mm, at least about 1 mm, at least about 1.5 mm, at least about 2 mm, at least about 2.5 mm, at least about 5 mm, at least about 7.5 mm, at least about 10 mm, at least about 15 mm, at least about 20 mm, at least about 25 mm, at least about 30 mm, at least about 35 mm, at least about 40 mm, or at least about 45 mm).

The hollow channel can have a width that ranges from any of the minimum dimensions to any of the maximum dimensions described above. For example, the hollow channel can have a width that ranges from about 0.1 mm to about 50 mm (e.g., from about 0.1 mm to about 25 mm, or from about 0.1 mm to about 10 mm). In some embodiments, the hollow channel can have widths of greater than 50 mm (e.g., as large as 1 cm).

In some embodiments, the hollow channel can have a height (e.g., defined as the distance between the floor and the ceiling of the hollow channel) of at least about 10 microns (e.g., at least about 25 microns, at least about 50 microns, at least about 75 microns, at least about 100 microns, at least about 150 microns, at least about 200 microns, at least about 300 microns, at least about 400 microns, at least about 500 microns, at least about 600 microns, or at least about 700 microns). In some embodiments, the hollow channel can have a height of about 750 microns or less (e.g., about 700 microns or less, about 600 microns or less, about 500 microns or less, about 300 microns or less, about 250 microns or less, about 200 microns or less, about 150 microns or less, about 100 microns or less, about 90 microns or less, about 80 microns or less, about 400 microns or less, about 300 microns or less, about 200 microns or less, about 150 microns or less, about 100 microns or less, about 75 microns or less, about 50 microns or less, or about 25 microns or less).

The hollow channel can have a height that ranges from any of the minimum dimensions to any of the maximum dimensions described above. For example, the hollow channel can have a height that ranges from about 10 microns to about 750 microns (e.g., from about 10 microns to about 500 microns, from about 10 microns to about 300 microns, from about 25 microns to about 300 microns, or from about 10 microns to about 75 microns).

The length of the hollow channel can be selected in view of a number of the overall device design and other operational considerations. In some embodiments, the hollow channel have a length of at least about 0.1 cm (e.g., at least about 0.2 cm, at least about 0.3 cm, at least about 0.4 cm, at least about 0.5 cm, at least about 0.6 cm, at least about 0.7 cm, at least about 0.8 cm, at least about 0.9 cm, at least about 1 cm, at least about 2 cm, at least about 2.5 cm, at least about 3 cm, at least about 4 cm, at least about 5 cm, or longer). The hollow channel in the microfluidic device can be substantially linear in shape, or they can possess one or more non-linear regions (e.g., a curved region, a spiral region, an angular region, serpentine, or combinations thereof) along the length of their fluid flow path. If desired for particular applications, three-dimensional networks of hollow channels can be fabricated.

The microfluidic devices described can be fabricated from any suitable material or combination of materials. In some embodiments, the devices are paper-based microfluidic devices. Paper-based microfluidic devices are microfluidic devices wherein the hollow channel for fluid flow is formed within one or more layers of a paper substrate material. An appropriate paper substrate material can be selected in view of the design of the device, the intended applications for the device, and considerations regarding device manufacture.

The paper substrate used to form the microfluidic device can be selected so as to be flexible. For certain applications, the paper substrate can be selected to have a sufficient flexibility such that the paper substrate can be folded, creased, or otherwise mechanically shaped to impart structure and function to the paper-based device. Examples of suitable paper substrates for the fabrication of paper-based microfluidic devices include cellulose; derivatives of cellulose such as nitrocellulose or cellulose acetate; paper (e.g., filter paper, chromatography paper); woven cellulosic materials; and non-woven cellulosic materials.

In some embodiment, the paper substrate is paper. Paper is inexpensive, widely available, readily patterned, thin, lightweight, and can be disposed of with minimal environmental impact. Furthermore, a variety of grades of paper are available, permitting the selection of a paper substrate with the weight (i.e., grammage), thickness and/or rigidity and surface characteristics (i.e., porosity, hydrophobicity, and/or roughness), desired for the fabrication of a particular paper-based device. Suitable papers include, but are not limited to, chromatography paper, card stock, filter paper, vellum paper, printing paper, wrapping paper, ledger paper, bank paper, bond paper, blotting paper, drawing paper, fish paper, tissue paper, paper towel, wax paper, and photography paper.

As described above, the floor, the two or more side walls, and/or optionally the ceiling can be substantially hydrophobic, so as to form boundaries that restrict fluid flow within the hollow channel, thereby defining a path for the flow of an aqueous solution through the hollow channel. In the case of paper-based devices, regions of the paper substrate forming the floor, the two or more side walls, and/or optionally the ceiling can be rendered hydrophobic (i.e., hydrophobically modified) by treating the paper substrate with a hydrophobic agent. For example, the paper substrate may be covalently modified to comprise a hydrophobic agent, impregnated with a hydrophobic agent, and/or coated with a hydrophobic agent to render portions of the paper substrate hydrophobic. Suitable hydrophobic agents include, but are not limited to, curable polymers, natural waxes, synthetic waxes, polymerized photoresists, alkyl ketene dimers, alkenyl succinic anhydrides, hydrophobic halosilanes, rosins, silicones, fluorinated reagents, fluoropolymers, polyolefin emulsions, resin and fatty acids, or combinations thereof. The hydrophobic agent can be patterned using methods known in the art to form hydrophobic regions of defined dimensions on and/or within the paper substrate, as required for the design of a particular device.

One or more of the interior surfaces of the hollow channel (e.g., the floor, a side walls, the ceiling, or a combination thereof) can comprise a hydrophilic material. The hydrophilic material can be porous or non-porous. In some embodiments, the hydrophilic material can comprise a hydrophilic coating deposited on an otherwise hydrophobic surface (e.g., a surface of a hydrophobically modified paper substrate) that forms the floor, a side walls, and/or a ceiling of the hollow channel.

In some cases, the hydrophilic material can be a porous hydrophilic material. For example, the hydrophilic material can comprise a region of a paper substrate described above that forms the floor, a side walls, and/or a ceiling of the hollow channel, and that has not been hydrophobically modified. The porous hydrophilic material can form a hemichannel for fluid flow in fluid contact with the hollow channel. Fluid can be transported through the porous hydrophilic hemichannel by capillary action, thereby driving fluid flow along the adjacent hollow channel.

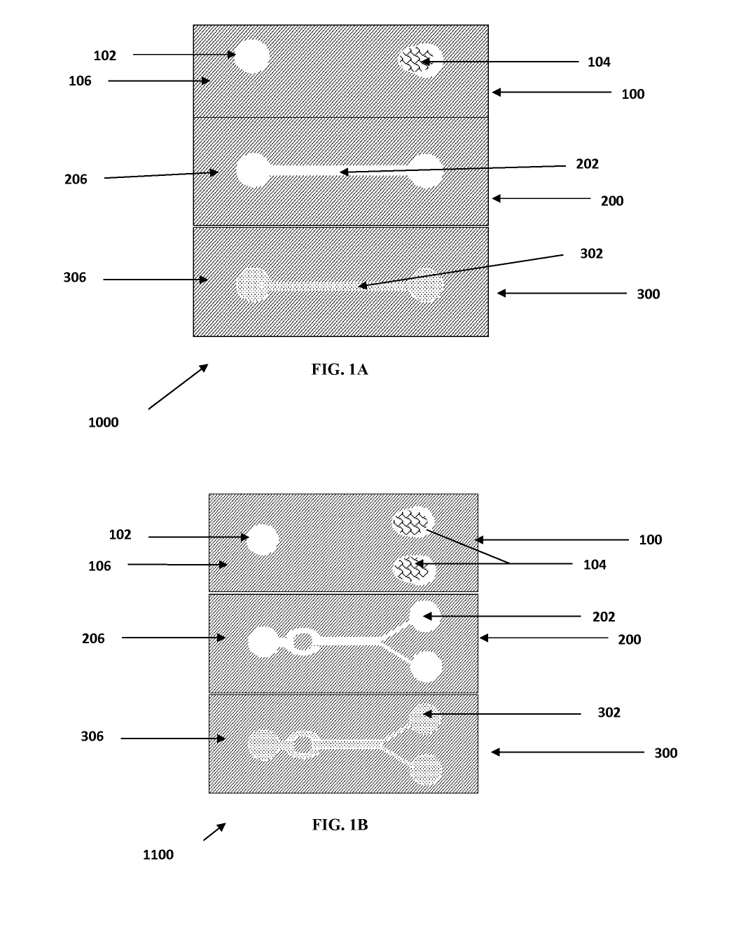

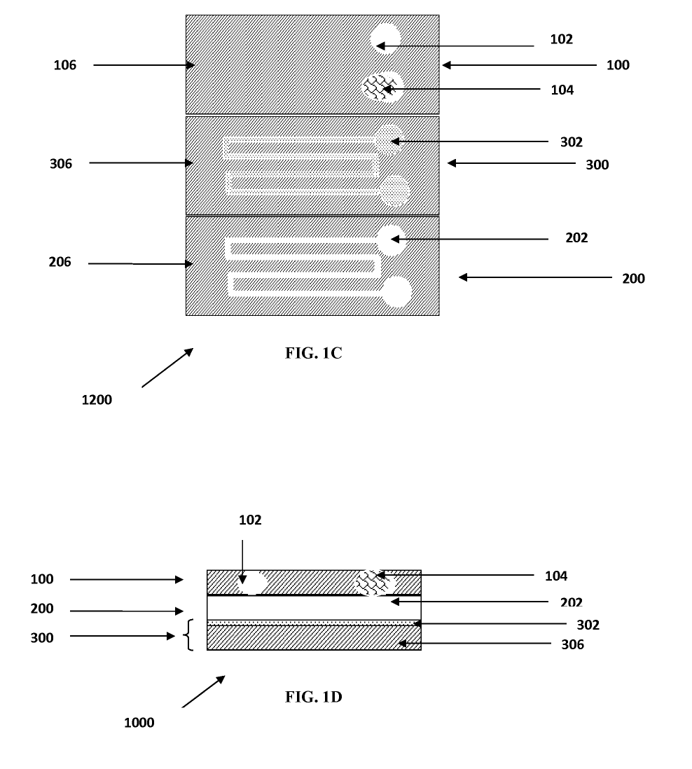

Example paper-based microfluidic devices including a hollow channel are schematically illustrated in FIGS. 1A-1D. Referring now to FIG. 1A, the device (1000) can include a sample deposition layer (100) having a top surface and a bottom surface, a channel layer (200) having a top surface and a bottom surface, and a base layer (300) having a top surface and a bottom surface. Referring now to FIG. 1D, the device can be assembled by stacking the three layers, such that when the device is assembled, the bottom surface of the sample deposition layer (100) is in contact with the top surface of the channel layer (200), and the bottom surface of the channel layer (200) is in contact with the top surface of the base layer (300).

Referring again to FIG. 1A, The sample deposition layer (100) can include a fluid inlet (102) defining a path for fluid flow from the top surface of the sample deposition layer to the bottom surface of the sample deposition layer, and a fluid outlet (104) defining a path for fluid flow from the bottom surface of the sample deposition layer to the top surface of the sample deposition layer. The fluid inlet (102) and the fluid outlet (104) can each comprise a region of porous hydrophilic material (e.g., a paper substrate that has not been hydrophobically modified). The fluid inlet (102) and the fluid outlet (104) can be delimited by one or more regions of hydrophobic material (106) that surround the fluid inlet (102) and/or the fluid outlet (104), and that substantially permeate the thickness of the paper substrate forming the sample deposition layer (100). The hydrophobic material (106) can thus define a path for fluid flow from one surface of the sample deposition layer to another surface of the sample deposition layer. The porous hydrophilic material forming the fluid inlet (102) and the fluid outlet (104) can be selected such that the fluid sample to be flowed through the hollow channel can be wicked through the fluid inlet (102) and the fluid outlet (104) by capillary action.

The channel layer (200) includes a hydrophobic boundary (206) defining a hollow channel (202) within the channel layer (200), for fluid flow within the second layer. The hydrophobic boundary (206) substantially permeates the thickness of the paper substrate, so as to form a boundary for fluid flow from the hollow channel to a region of the channel layer outside of the channel.

The hollow channel can be patterned within a channel layer formed from a paper substrate using any suitable method known in the art. For example, the channel can be patterned by wax printing. In these methods, an inkjet printer is used to pattern a wax material on the paper substrate. Many types of wax-based solid ink are commercially available and are useful in such methods as the ink provides a visual indication of the location of the hollow channel. However, it should be understood, that the wax material used to form the channel does not require an ink to be functional. Examples of wax materials that may be used include polyethylene waxes, hydrocarbon amide waxes or ester waxes. Once the wax is patterned, the paper substrate is heated (e.g., by placing the substrate on a hot plate with the wax side up at a temperature of 120° C.) and cooled to room temperature. This allows the wax material to substantially permeate the thickness of the paper substrate, so as to form a hydrophobic boundary that defines the dimensions of the hollow channel. The hollow channel can then be formed by removing the porous, cellulosic substrate within the hydrophobic boundary, thereby forming a void space through which a fluid can flow.

Referring again to FIG. 1A, the base layer of the device (300) can include a top surface, a bottom surface, and hydrophilic material (302) patterned on or within the base layer, such that the hydrophilic material can form at least a portion of the top surface of the base layer. The dimensions of the hydrophilic material (302) can be delimited by one or more regions of hydrophobic material (306). The hydrophilic material (302) can comprise a region of a paper substrate that has not been hydrophobically modified. In these embodiments, the porous hydrophilic material can form a hemichannel for fluid flow. Fluid can be transported through the porous hydrophilic hemichannel by capillary action, thereby driving fluid flow along the adjacent hollow channel.

The example device can be assembled by aligning the three layers as shown in FIG. 1D. The sample deposition layer (100), the channel layer (200), and the base layer (300) are stacked such that the bottom surface of the sample deposition layer (100) is in fluid communication with the top surface of the channel layer (200), and the bottom surface of the channel layer (200) is in fluid communication with the top surface of the base layer (300). When stacked, the sample deposition layer (100), the channel layer (200), and the base layer (300) are aligned so as to form a path for fluid flow from the fluid inlet (102) through the hollow channel (202) to the fluid outlet (104). The fluid flow path of the hollow channel (202) in the channel layer (200) is defined by a floor formed from the top surface of the base layer (300), two side walls formed by the hydrophobic boundary (206) of the channel layer (200), and a ceiling formed from the bottom surface of the sample deposition layer (100). When stacked, the channel layer (200) and the base layer (300) are aligned such that the hydrophilic material (302) forms at least a portion of the floor of the hollow channel (202).

Fluid flow through the hollow channel can be driven by a combination of pressure applied to the fluid inlet and/or fluid outlet, capillary flow through and/or along the hydrophilic material, and the hydrophobicity of the interior surfaces of the hollow channel. In some embodiments, fluid flow in the hollow channel is driven by capillary flow through and/or along a hydrophilic material present in the fabricated into a portion of the periphery of the hollow channel. In some embodiments, the hollow channel can be configured such that water can flow from the fluid inlet through the hollow channel to the fluid outlet under low applied pressure to fluid introduced at the fluid inlet (e.g., at an applied pressure 0.2 bar or less, at an applied pressure of 0.1 bar or less, at an applied pressure of 0.05 bar or less, or at an applied pressure of 0.01 bar or less). In some embodiments, fluid can flow from the fluid inlet through the hollow channel to the fluid outlet without the aid of pressure applied by an external pump (e.g., a syringe pump) and/or a column of fluid positioned to applied pressure at the fluid inlet.

The microfluidic devices described herein can optionally comprise one or more additional elements, as required to provide a device with suitable functionality for a particular application. For example, the microfluidic device can optionally comprise one or more additional layers, such as a slip layer. The slip layer can configured such that actuation of the slip layer can slow or stop the flow of a fluid through the hollow channel. The slip layer can configured such that actuation of the slip layer can introduce an assay reagent, discussed in more detail below, into contact with a fluid flowing through the hollow channel. A slip layer may be disposed between the sample deposition layer and the channel layer and/or between the channel layer and the base layer.

In some embodiments, the microfluidic device can further comprise an assay reagent to aid in detection and/or quantification of an analyte present in a fluid sample flowing through the hollow channel. By way of example, the analyte can be a molecule of interest present in a fluid sample that is introduced into the channel. The analyte can be, for example, an antibody, peptide (natural, modified, or chemically synthesized), protein (e.g., a glycoprotein, a lipoprotein, or a recombinant protein), polynucleotide (e.g, DNA or RNA, an oligonucleotide, an aptamer, or a DNAzyme), lipid, polysaccharide, small molecule organic compound (e.g., a hormone, a prohormone, a narcotic, or a small molecule pharmaceutical), pathogen (e.g., bacteria, virus, or fungi, or protozoa), or combination thereof.

The fluid sample can be a bodily fluid. “Bodily fluid”, as used herein, refers to a fluid composition obtained from or located within a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples. The molecule of interest can be, for example, a biomarker (i.e., a molecular indicator associated with a particular pathological or physiological state) present in the bodily fluid that can be assayed to identify risk for, diagnosis of, or progression of a pathological or physiological process in a subject. Examples of biomarkers include proteins, hormones, prohormones, lipids, carbohydrates, DNA, RNA, and combinations thereof.

The assay reagent can include a molecule or matrix that can selectively associate with the analyte. The term “selectively associates”, as used herein when referring to an assay reagent, refers to a binding reaction which is determinative for the analyte in a heterogeneous population of other similar compounds. Generally, the interaction is dependent upon the presence of a particular structure (e.g., an antigenic determinant or epitope) on the binding partner. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism. Examples of such molecules include antibodies, antibody fragments, antibody mimetics (e.g., engineered affinity ligands such as AFFIBODY® affinity ligands), peptides (natural or modified peptides), proteins (e.g., recombinant proteins, host proteins), polynucleotides (e.g, DNA or RNA, oligonucleotides, aptamers, or DNAzymes), receptors, ligands, antigens, organic small molecules (e.g., antigen or enzymatic co-factors), and combinations thereof. In some embodiments, the assay reagent can include a probe selected to facilitate radiological, magnetic, optical, and/or electrical measurements used to identify and/or quantify one or more analytes in a liquid sample. For example, the assay reagent can include a colorimetric probe, a fluorescent probe, a probe to facilitate electrochemical detection and/or quantification of an analyte, or combinations thereof, as discussed in more detail below.

The assay reagent can be positioned in fluid contact with the hollow channel, such that, as fluid migrates through the flow path of the hollow channel toward the fluid outlet, the assay reagent contacts the analyte. The assay reagent can also be deposited, for example on the fluid inlet and/or fluid outlet of the device, and/or at one or more assay regions in fluid contact with the fluid flow path. Assay reagents can be deposited in discrete areas, using e.g. a micro-arraying tool, ink jet printer, spray, pin-based contact printing or screen-printing method.

In some embodiments, the microfluidic device may contain one or more assay regions containing one or more assay reagents selected so as to provide a response in the presence of an analyte that is visible to the naked eye. In some cases, the assay reagent can be an indicator that exhibits colorimetric and/or fluorometric response in the presence of the analyte of interest. Indicators may include molecules that become colored in the presence of the analyte, change color in the presence of the analyte, or emit fluorescence, phosphorescence, or luminescence in the presence of the analyte. In these embodiments, the presence of an analyte may be ascertained by simple visual examination, optionally under a blacklight. In some cases, the quantity of one or more analytes may be determined by visual inspection of the color or fluorescence of an assay region, for example, by comparison to known colors at predetermined analyte concentrations.

Alternatively, the devices described herein can include a detection device that can evaluate the fluid sample and/or the assay reagent to indicate, for example, the presence, identity, or quantity of an analyte in a fluid sample. For example, a microfluidic device may contain one or more fluid outlets that connect the device to one or more external instruments, such as a mass spectrometer, fluorometer, LTV-Vis spectrometer, IR spectrometer, gas chromatograph, gel permeation chromatograph, DNA sequencer, Coulter counter, or combinations thereof, that can be used to analyze the fluid sample processed by the device. The microfluidic device can optionally be configured such that the fluid sample and/or assay reagent can be interrogated using a portable device, such as a digital camera, flatbed scanner, or cellular phone.

In certain embodiments, detection and/or quantification of the analyte can be accomplished using electrochemical methods. In some embodiments, the microfluidic device can comprise an electrode in electrochemical contact with the hollow channel, meaning that the electrode can participate in a faradaic reaction with one or more components of a fluid present in the hollow channel of the microfluidic device. For example, the electrode can be configured such that a surface of the electrode is in direct contact with fluid present in the hollow channel of the microfluidic device. The device can be configured such that the electrode can function as an anode, cathode, or anode and cathode during device operation.

The electrode can be configured to provide detection of an analyte or molecule of interest. For example, the device can include a three electrode system comprising a working electrode (analyte working electrode), a counter electrode, and a reference electrode (either a conventional reference electrode or a pseudo reference electrode). All three electrodes can be positioned in electrochemical contact with a region of the hollow channel within the microfluidic device.

Electrodes can be fabricated from any suitable conductive material, such as a metal (e.g., gold, platinum, or titanium), metal alloy, metal oxide, conducting polymer (e.g., PEDOT or PANI), or conductive carbon. The electrodes can be, for example, screen printed electrodes formed using a conductive ink. In certain embodiments, the electrode can be a bulk electrode. The bulk electrode can have a variety of 3-dimensional shapes, provided that the electrode can be integrated into the device, and is compatible with the formation of an electric field gradient suitable to direct ions flowing through the device. In certain embodiments, the bulk electrode is a bulk conductive electrode. Suitable bulk conductive electrodes include, but are not limited to wire, mesh, fiber, plate, foil, perforated plate, and perforated foil metal electrodes.

The devices described herein can be coupled to a power supply and optionally to one or more additional suitable features including, but not limited to, a voltmeter, an ammeter, a multimeter, an ohmmeter, a signal generator, a pulse generator, an oscilloscope, a frequency counter, a potentiostat, or a capacitance meter. The devices described herein can also be coupled to a computing device that performs arithmetic and logic operations necessary to process the electrochemical signals produced by the device (e.g., to determine analyte concentration, etc.).

The devices described herein can optionally further comprise structures that influence fluid flow through the hollow channel, manipulate the fluid sample as it flows through the hollow channel, and/or enhance or make more frequent the contact of analytes in solution with an assay reagent. For example, the device can include one or more obstacles disposed in the hollow channel to slow or stop the flow of a fluid through the hollow channel. Examples of suitable obstacles include pillars, beads, paper barriers, hydrophobic weirs, and combinations thereof. In some embodiments, the structures can be stimuli responsive. For example, the structures can be chemically or photonically responsive. In some embodiments, the structure can be a barrier that is present in the device when a fluid sample is first introduced into the device, but is removed at a later point upon application of a stimulus. For example, the structure can be a barrier that is present in the device when a fluid sample is first introduced into the device, but that dissolves at a later point (e.g., a photonically activated barrier that depolymerizes upon incident light, or a chemically activated barrier that reacts and/or dissolves upon contact with a particular chemical).

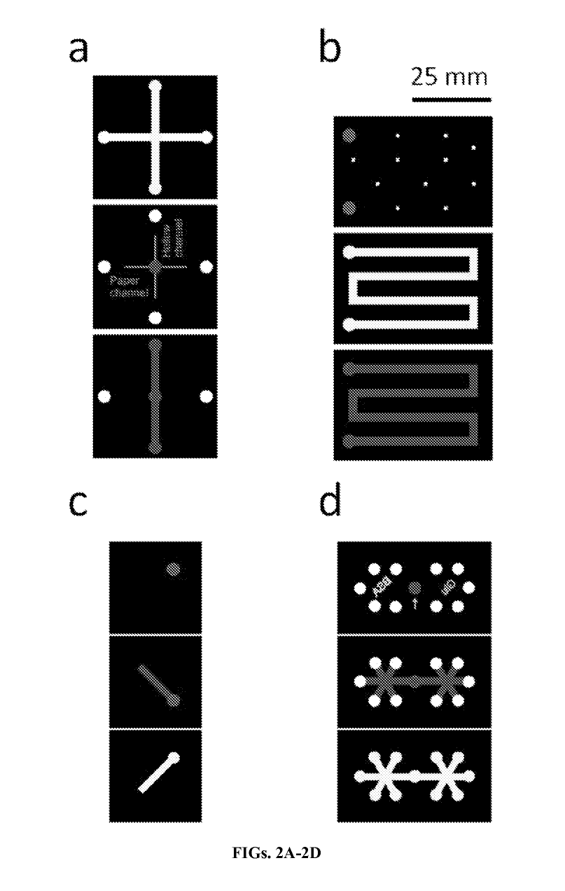

The microfluidic device can comprise a plurality of hollow channel. For example, for determining multiple analytes, the device may contain a plurality of hollow channels that can be used to process a fluid sample. These may be arranged in parallel or in any other convenient manner. Each of the plurality of hollow channels can contain an assay reagent for different analyte of interest. By way of example, FIG. 2D is an illustration of a microfluidic device that includes multiple hollow channels. The example device comprises a single fluid inlet fluidly connected to two hollow channels, each of which leads to a plurality of fluid outlets. Each of the fluid outlets can include an assay reagent (and thus serve as an assay region for an individual analyte of interest). In this way, a single fluid sample can be rapidly and simultaneous screened for a number of analytes.

If desired, the devices described herein can be affixed to or secured within a polymer, metal, glass, wood, or paper support structure to facilitate handling and use of the device. In some embodiments, the devices described herein are affixed to or secured within an inert, non-absorbent polymer such as polydimethylsiloxane (PDMS), a polyether block amide (e.g., PEBAX®, commercially available from Arkema, Colombes, France), a polyacrylate, a polymethacrylate (e.g., poly(methyl methacrylate)), a polyimide, polyurethane, polyamide (e.g., Nylon 6,6), polyvinylchloride, polyester, (HYTREL®, commercially available from DuPont, Wilmington, Del.), polyethylene (PE), polyether ether ketone (PEEK), fluoropolymers such as polytetrafluoroethylene (PTFE), perfluoroalkoxy, fluorinated ethylene propylene, or a blend or copolymer thereof. Silastic materials and silicon-based polymers can also be used.

Methods of Use

The devices described herein can be inexpensive, user friendly (e.g., they can employ electrochemical detection without any washing steps), sensitive, portable, robust, efficient, rapid, and can be used to detect low concentrations of analytes. As such, the devices are well suited for use in numerous analytical applications. For example, the devices described herein can be used in clinical and healthcare settings to detect and/or quantify biomarkers to identify risk for, diagnosis of, or progression of a pathological or physiological process in a subject. Examples of biomarkers include proteins, hormones, prohormones, lipids, carbohydrates, DNA, RNA, and combinations thereof.

The devices described herein can be used in POC applications to diagnose infections in a patient (e.g., by measuring serum antibody concentrations or detecting antigens). For example, the devices can be used for amperometric and potentiometric detection of glucose, lactate, uric acid, ascorbic acid, β-D-galactosidase, cholesterol, Pb²⁺, H₂O₂, and cancer markers. In some embodiments, the devices described herein can be used to diagnose viral infections (e.g., HIV, hepatitis B, hepatitis C, rotavirus, influenza, polio, measles, yellow fever, rabies, dengue, or West Nile Virus), bacterial infections (e.g., E. coli, C. tetani, cholera, typhoid, diphtheria, tuberculosis, plague, Lyme disease, or H. pylori), and parasitic infections (e.g., toxoplasmosis, Chagas disease, or malaria). The devices described herein can be used to rapidly assesses the immune status of people or animals against selected vaccine-preventable diseases (e.g. anthrax, human papillomavirus (HPV), diphtheria, hepatitis A, hepatitis B, haemophilus influenzae type b (Hib), influenza (flu), Japanese encephalitis (JE), measles, meningococcal, mumps, pertussis, pneumococcal, polio, rabies, rotavirus, rubella, shingles (herpes zoster), smallpox, tetanus, typhoid, tuberculosis (TB), varicella (chickenpox), yellow fever). The devices described herein can be used to rapidly screen donated blood for evidence of viral contamination by HIV, hepatitis C, hepatitis B, and HTLV-1 and -2. The devices described herein can also be used to measure hormone levels. For example, the devices and methods described herein can be used to measure levels of human chorionic gonadotropin (hCG) (as a test for pregnancy), Luteinizing Hormone (LH) (to determine the time of ovulation), or Thyroid Stimulating Hormone (TSH) (to assess thyroid function). The devices described herein can be used to diagnose or monitor diabetes in a patient, for example, by measuring levels of glycosylated hemoglobin, insulin, or combinations thereof. The devices and methods described herein can be used to detect protein modifications (e.g., based on a differential charge between the native and modified protein and/or by utilizing recognition elements specific for either the native or modified protein). The devices described herein can be used to administer personalized medical therapies to a subject (e.g., in a pharmacogenomic assay performed to select a therapy to be administered to a subject). The devices can also be used to monitor the vascular endothelial growth factor (VEGF) levels in the urine of infants, e.g., premature infants. A conventional method of diagnosing retinal disease in premature infants is weekly or biweekly 15 minute examinations by an infant-retinal ophthalmologist, which is both expensive and disruptive to the infant. Detecting VEGF and other growth factors (such as IGF-1, or insulin-like growth factor 1) in urine can be useful for diagnosing retinopathy of prematurity, diabetes, cancer, and transplantation.

In other embodiments, the device can be used to analyze cerebrospinal fluid (CSF), for example to determine whether a patient has meningitis. In some embodiments, the devices can be used for breast milk analysis, e.g., to determine protein, fat, and glucose levels in the breast milk. In other embodiments, the devices can be used in tissue engineering applications, to monitor the output of small numbers of cells, e.g., measuring albumin output from small cultures of hepatocytes. Catalytic chemistries, such as ELISA, can be incorporated into the devices in order to make measurements of relatively small specimens. In still other embodiments, the devices can be used in ophthalmology, e.g., in analyzing components in the vitreous fluid (the contents of the eye) or in tear films.

The devices described herein can also be used in other commercial applications. For example, the devices described herein can be used in the food and beverage industry, for example, in quality control applications or to detect potential food allergens, such as milk, peanuts, walnuts, almonds, and eggs. The devices described herein can be used to detect and/or measure the levels of proteins of interest in foods, cosmetics, nutraceuticals, pharmaceuticals, and other consumer products. The devices described herein can also be used to rapidly and accurately detect narcotics and biothreat agents (e.g., ricin).

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

### Example 1: Hollow-Channel Paper Analytical Devices (HC-PADs)

A microfluidic paper analytical device (μPAD) that relies on flow in hollow channels (HC) to transport fluids was fabricated. The flow rate of a solution in the hollow channel is enhanced by up to a factor of 7 relative to fluid flow rate through a paper-filed channel of the same dimensions. The HC-PAD does not require external equipment, such as a syringe pump, to force the liquid into the channel. The high flow rate of the liquid reduces analysis times and also makes it possible to use large fluidic networks. The microfluidic device can multiplex numerous assays without being compromised by the speed of fluid flow.

Materials and Methods

Chemicals and Materials.

Erioglaucine disodium salt was purchased from Acros Organics. Phosphate-buffered saline (PBS), 10× solution, 30% HCl, and Whatman grade 1 chromatographic paper were obtained from Fisher Scientific. Glucose oxidase (GOx) from Aspergillus niger (type X-S), peroxidase from horseradish (type VI) (HRP), D-(+)-glucose (referred to as glucose), and albumin from bovine serum (BSA) were purchase from Sigma-Aldrich. Tetrabromophenol blue was obtained from Alfa Aesar. Sodium citrate was provided by EM Science. KI was obtained from Mallinckrodt Specialty Chemicals Co., and ethanol (99.5%) was purchased from Pharmaco-Aaper. All solutions were prepared using deionized water (18.2 MΩ·cm, Milli-Q Gradient System, Millipore). All reagents were used as received without further purification.

Device Fabrication.

The PADs were fabricated using a previously reported wax patterning method (Lu, Y. et al., Electrophoresis 2009, 30, 1497-500; Carrilho, E. et al., Anal. Chem. 2009, 81, 7091-7095). The devices were designed using CorelDraw12 software, and the specific patterns used for the different paper devices are shown in FIGS. 2A-2D. Patterns were printed on Whatman chromatographic paper using a Xerox 8570DN inkjet wax printer. The paper was then placed in an oven at 120° C. for 1 min and then cooled to 20° C. The paper channels and reservoirs were cut using, respectively, a razor blade and a 4 mm inner-diameter punch (Harris Uni-core). Sharp tools were used to obtain a clean cut and to avoid clogging the channels.

Glucose and BSA Assays.

For the glucose and BSA assays, the reagents were dried in paper reservoirs defined on the top layer of the device. Finally, the PAD was folded according to the origami technique and tightly pressed together using two rigid 5 mm thick-polycarbonate pieces clamped with binder clips.

The glucose assay was prepared as follows. First, 1.0 μL of 0.86 M KI was drop casted into the paper wells. Second, after the KI solution was dried, 1.0 μL of a horseradish peroxidase/glucose oxidase solution (20/100 units) in PBS solution 1× (12 mM phosphate buffer, pH 7.4, 137 mM NaCl, and 2.7 mM KCl) was added to the wells. The BSA assay was prepared by drying 0.5 μL of a 250 mM citrate solution (sodium citrate solution acidified with concd HCl, pH 1.7) into each well, followed by addition of 0.5 μL of 3.3 mM tetrabromophenol blue in 95% ethanol. The solutions were dried at 20° C. under N₂. The glucose standards were prepared by diluting a glucose stock solution in PBS 1× buffer. The glucose stock solution was prepared 1 day before the experiment to allow the glucose to mutarotate. The BSA standards were also prepared in PBS 1× buffer. An once scanner (HP C6180) was used to acquire optical images of the paper devices, and ImageJ freeware (NIH, Bethesda, Md.) was used to analyze the colors. For the glucose assay, the color pictures were converted to grayscale, and then the average intensity was correlated to the concentration of glucose. For the BSA assay, each pixel of the picture was split into red, green, and blue color spaces. The color intensity of the red channel was correlated with the concentration of BSA.

Results and Discussion

Fast Liquid Transport in Hollow Channels.

The flow rate of an aqueous solution of a blue dye in a hollow channel as a function of time and pressure using the configuration shown in FIG. 4A was investigated. The location of the dye was established by observing the passage of 5.0 mM aqueous erioglaucine past unwaxed 300 μm diameter paper windows defined along the hollow channel. The pressure was controlled by varying the height of the dye solution in the inlet reservoir. Because the height of the solution in the reservoir varies by a maximum of 10% during the time required to run experiments, the pressure was nearly constant for each measurement. The pressure P at the inlet was calculated using eq 1. Here ρ is the density of water

P=ρ×g×h (1)

at 20° C., g is the gravitational constant, and h is the height of the liquid in the inlet reservoir. To fully evaluate the performance of hollow channels, control experiments with paper channels were also carried out.

FIG. 4B compares the distances traveled by an aqueous dye solution in hollow and paper channels during a 70 s time interval. The instantaneous flow rate, calculated as the derivative of distance as a function of time, is shown in FIG. 4C. For both hollow and paper channels, the flow rate is not constant and decreases with time. However, the liquid flows much faster in the hollow channel. Indeed, during the 70 s duration of the experiment the flow rate in the hollow channel is on average 7 times higher than for the paper channel. This means that the dye travels about 12 cm in 70 s, compared to just 2 cm in the paper channel. Note that it takes about 1 h for the solution to flow 12 cm in a paper channel, and during this period evaporation of the sample can become a major problem.

The effect of the pressure on the flow rate is shown FIG. 4D. The pressure at the inlet was varied from 1.2 to 2.7 mbar. Higher pressures lead to faster flow rates in hollow channels, but they have no effect on the flow rate in paper channels as demonstrated by the two superimposed curves (1.2 and 2.7 mbar) at the bottom of FIG. 4D. The pressure of a single drop of liquid, exerting ˜0.2 mbar of pressure, is sufficient to fill a hollow channel. In fact, the pressure of a single drop is sufficient to fill a 1.5 cm long hollow channel in ˜2 s while it takes 30 s to fill a paper channel having the same dimensions.

Although the high flow rates observed in hollow channels are primarily driven by pressure, capillary flow may also be important depending on the degree of hydrophobicity of the channel walls. In the absence of a hydrophilic floor, aqueous solutions do not enter inside the hollow channel over the pressure range represented in FIG. 4D. This finding is consistent with results recently reported by Glavan et al. (Lab Chip, 2013), wherein they use a syringe pump or relatively high pressures (˜200 mbar, i.e., a column of water 2 m high) to drive fluids through hydrophobic paper channels. The hydrophilic floor enables low-pressure (i.e., 0.2 mbar), high-speed flow through hollow channels.

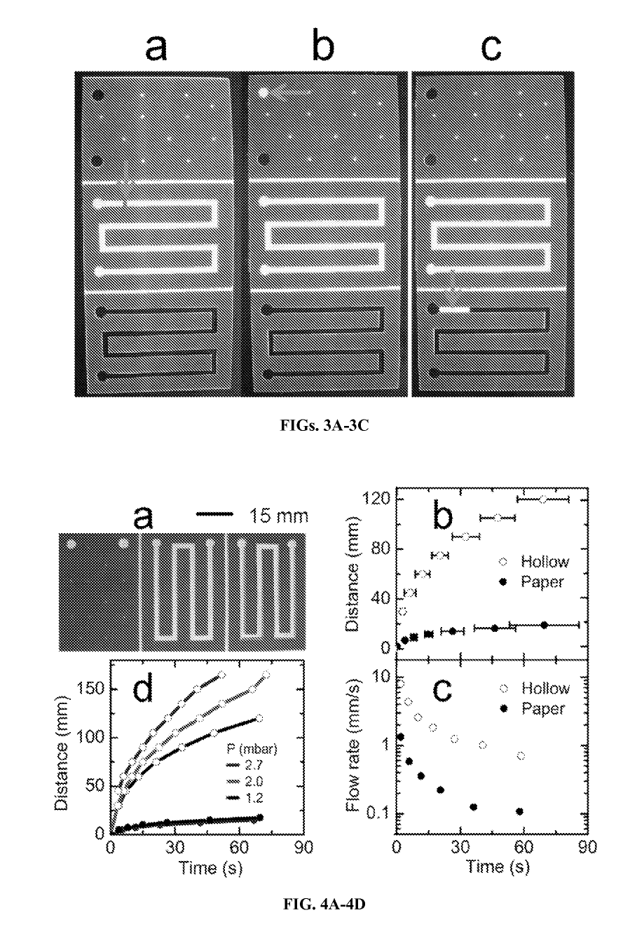

As alluded to by the results presented thus far, the flow of liquids in hollow channels can be conveniently controlled by adjusting pressure and capillary forces. Indeed, in the absence of obstacles within the hollow channel, the liquid quickly reaches the outlet of the device and continues to flow until the inlet reservoir is empty. However, if there is a paper barrier within the hollow channel, the associated flow resistance can slow down the liquid, or stop it entirely, depending on the length of the barrier and the pressure at the inlet. For example, a 180 μm-long paper barrier placed at the inlet decreases the flow rate by a factor of 2 (compared to a barrier-free channel) under the influence of a 1.2 mbar pressure at the inlet. However, a 1 cm-long paper barrier completely stops the pressure-driven flow, leaving only the hydrophilic floor wet. Moreover, a 1 mm wide-hydrophobic wax line perpendicular to the hollow channel completely stops the liquid. The important point is that, in analogy to constrictions within other types of microfluidic devices, wax lines and paper barriers can be used to control flow rates from between 0 and several mm/s. Photographs of the devices showing the precise location of the barriers used for the aforementioned experiments are provided in FIG. 3.

Colorimetric Detection of Glucose and BSA.

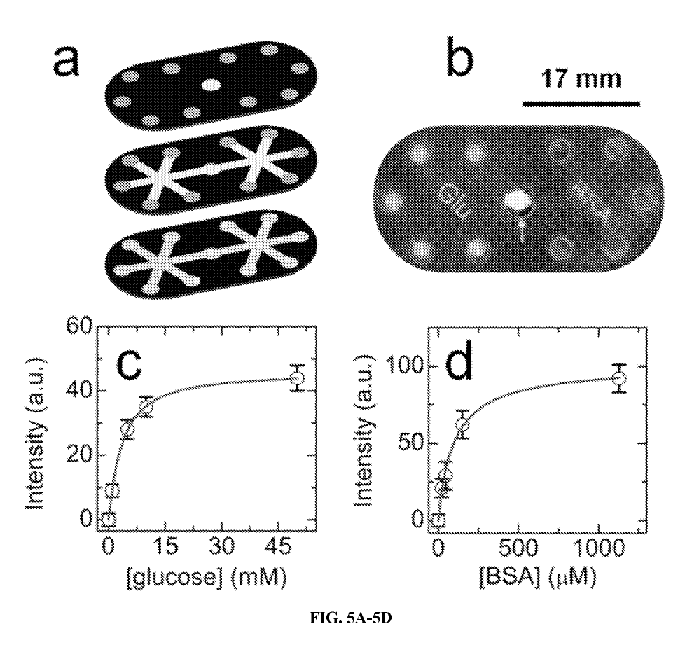

To demonstrate the potential of hollow channels for carrying out simple assays, glucose and BSA colorimetric reactions were used. The multiplexed assay was carried out using the 3D PAD design shown in FIG. 5A. The paper was prepared by drying the reagents for the glucose and BSA assays in the paper wells on the top layer of the device. The five wells on left and right were filled with the assay reagents for glucose and BSA, respectively. After the reagents were dried, the device was folded and an 80 μL drop of sample was introduced at the inlet located at center of the device. The hollow channel network shown in FIG. 5A directs the sample toward the two separate test zones. For both assays, the sample is split and delivered into each of five different wells to achieve five replicate results. Five minutes after the injection of the sample, excess liquid was removed from the inlet, and then the device was scanned to quantify the color change in the test wells.

A photograph of the paper device 5 min after injection of a sample containing 20 mM of glucose and 75 μM of BSA is shown FIG. 5B. A change of color in both the glucose and BSA testing wells is easily detected by the naked eye, which provides a means for making a quick semiquantitative reading. More importantly, however, quantitation can be achieved by analyzing the change of color using a desktop scanner. Samples containing different concentrations of glucose and BSA were used to calibrate the PAD, and the resulting calibration curves for glucose and BSA are plotted in FIGS. 5C and 5D, respectively. A power function was used to fit the data, and the limits of detection (LODs) were found to be 0.7 mM for glucose and 18 μM for BSA.

For the PAD assay described in the previous paragraph, it takes about 0.5 min for the sample to flow from the inlet to the reaction wells. For the 5 min total assay time, this leaves 4.5 min to develop the color in the test zones. For a paper device having a similar design, but paper rather than hollow channels, it takes ˜2 min for the sample to reach the test zones. Thus, while sample transport accounts for only 10% of the total assay time in the hollow-channel PAD, it consumes 40% of the assay time in a paper channel. Note that the more complex or multiplexed the assay, the more advantage there is to the hollow channels. Additionally, the larger-than-usual footprint of the PAD used for the glucose and BSA assays (3.4×2.0 cm) is easier to handle than smaller paper-channel-based PADs, which is an important point for some POC applications.

Summary

Hollow channels enable fluid transport in paper-based devices up to 7 times faster than in cellulose-containing channels. The results indicate that flow is induced by a single drop of sample, thereby avoiding the need for pumping equipment. The flow rate within the hollow channels can be controlled by inserting hydrophobic weirs or short cellulose sections. Paper-based PADs having cellulose channels for DNA assays showed moderate to severe NSA even in the presence of blockers, a problem largely avoided by using hollow channels.

### Example 2: Electrochemistry and Mass Transfer in Hollow-Channel Paper Analytical Devices

This example analyses electrochemical and fluidic processes in paper-based analytical devices (PADs) having hollow channels (HC-PADs). The HC-PADs exhibit electrochemical and hydrodynamic behavior similar to traditional glass and plastic microfluidic electrochemical devices. Removal of the cellulose fibers from the channels results in rapid mass transfer. The flow rate within the channel was quantified by electrochemical methods for pressures ranging from 0.3 mbar to 4.5 mbar. Voltammetry and amperometry were applied under flow and no-flow conditions and yielded reproducible electrochemical signals that can be described by classical electrochemical theory as well as finite-element simulations. The results shown here provide new and highly quantitative insights into the mass transfer and electrochemical properties of HC-PADs.

Materials and Methods

Chemicals and Materials.

Ferrocenemethanol (FcMeOH), and 1,1′-ferrocenedimethanol (FcDM) were purchased from Sigma-Aldrich (St. Louis, Mo.). Whatman grade 1 chromatography paper (20 cm×20 cm sheets), NaCl, and concentrated pH 7.4 phosphate buffered saline solution (PBS 10×, 119 mM phosphate, 1.37 M NaCl and 27 mM KCl) were purchased from Fisher Scientific (Waltham, Mass.). Tris(1,10-phenanthroline) iron(II) sulfate (Fe(phen)3SO4) and resazurin were purchased from Acros Organics (Morris Plains, N.J.). Tartrazine was purchased from MP Biomedicals LLC (Solon, Ohio). 4′,6-diamidino-2-phenylindole, dihydrochloride (DAPI) was purchased from Life Technologies (Carlsbad, Calif.). The carbon (CI-2042) and Ag(83%)/AgCl(17%) (CI-4002) inks were purchased from Engineered Conductive Materials (Delaware, Ohio). The solutions were prepared using deionized water (18.2 MΩ·cm, Milli-Q Gradient System, Millipore). All chemicals were used as received.

Device Fabrication.

The HC-PADs were fabricated using a previously reported wax patterning method (Renault, C. et al., Anal. Chem., 2013, 85, 7976-7979). The patterns were designed using CorelDraw12 software and printed on Whatman grade 1 chromatographic paper using a Xerox 8570DN inkjet wax printer. The patterns used for the different paper devices are shown in FIG. 13. After printing, the paper was placed in an oven at 125° C. for 1 min to melt the wax, and then it was cooled to 23° C. The carbon and Ag/AgCl electrodes were screen-printed directly on the paper devices using a mesh with 305 threads per inch² (Ryonet Corporation, Vancouver, Wash.).

The inks were then cured in an oven at 65° C. for 30 min. Channels and reservoirs larger than 2 mm were cut using a razor blade and a 4 mm-diameter punch (Harris Uni-core), while smaller HCs were cut using a laser cutter (Epilog Zing 16 from Epilog Laser, Golden, Colo. Parameters: Vector image, Speed: 90%, Power: 10%, Frequency: 1500 Hz). In all cases, clean cuts are required to avoid clogging the channels. After cutting the channels, the paper was folded into the final device configuration, sandwiched between two rigid, 5 mm-thick poly(methyl methacrylate) holders, and then clamped with binder clips. Copper tape (3M) was used to establish contact between the screen-printed electrodes and the potentiostat.

Electrochemical Measurements.

Electrochemical measurements were carried out at room temperature (23±1° C.) using a potentiostat (650 C, CH Instruments, Austin, Tex.) or bipotentiostat (700 E, CH Instruments, Austin, Tex.). In some cases a Pt wire counter electrode and a reference electrode (either a glass Ag/AgCl, 1 M KCl or a saturated calomel electrode (SCE), CH Instruments, Austin, Tex.) were placed into the outlet reservoir of the HC-PAD. The ohmic resistance in the HCs was electronically compensated (R_{comp}).

Numerical Simulations.

Numerical simulations were performed using a Dell Precision T7500 Simulation workstation outfitted with Dual Six Core Intel Xeon Processors (2.40 GHz) and 24 GB of RAM. Simulations were carried out using the COMSOL Multiphysics v4.3 commercial package. All simulations were performed in 2D. Convective models solved the Navier-Stokes equation, assuming an incompressible fluid and no-slip boundary conditions on the floor and ceiling. Convection-diffusion simulations were performed assuming that the concentration of analyte was zero at the electrode surface, corresponding to the mass-transfer limited case.

Results and Discussion

Electrochemistry in Absence of Convection.

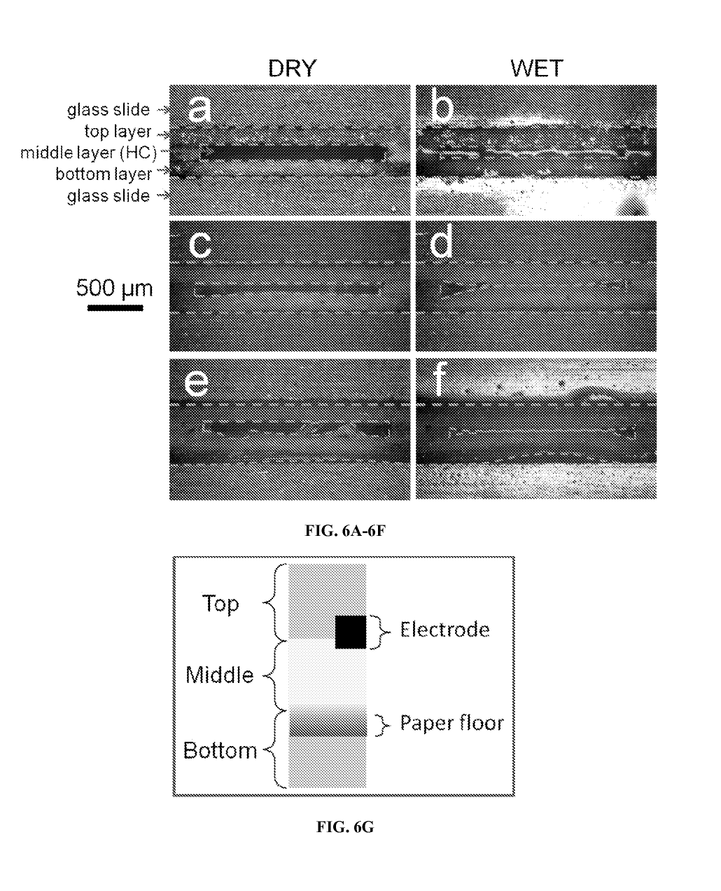

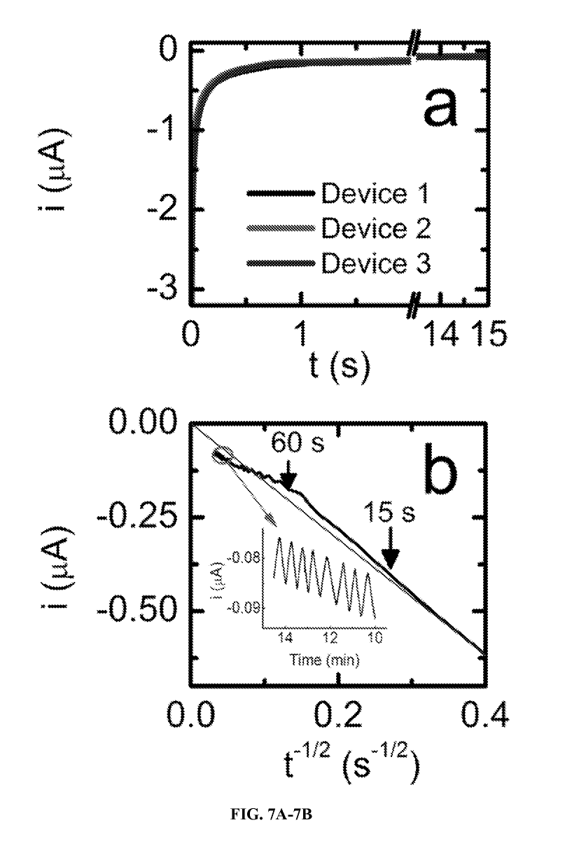

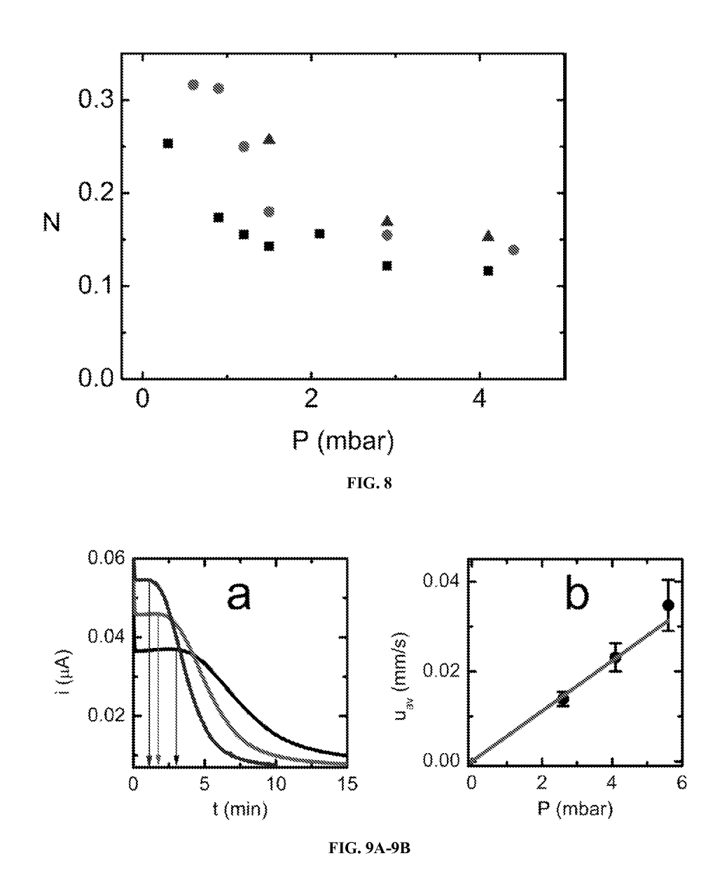

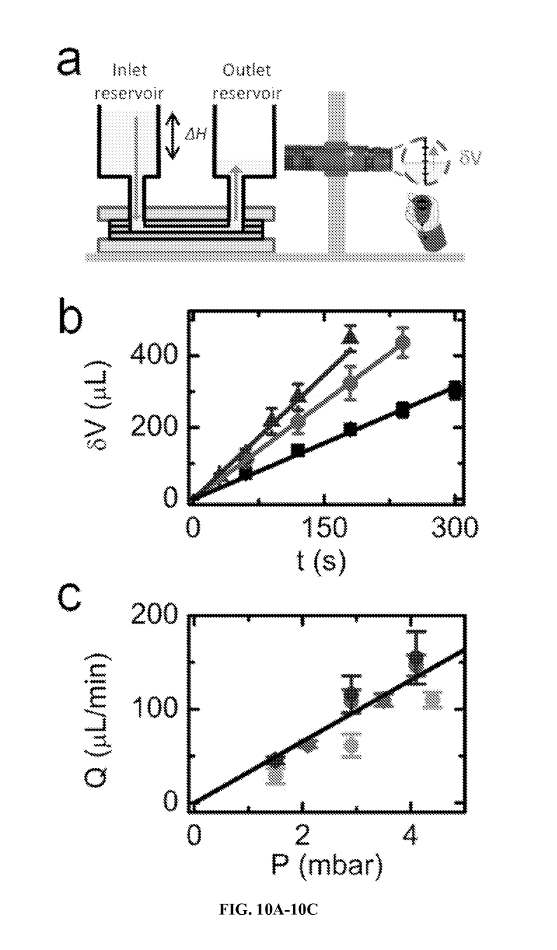

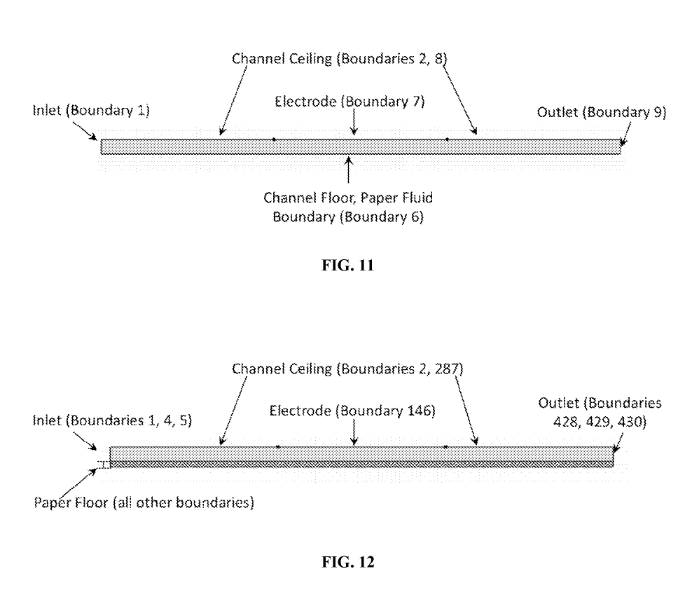

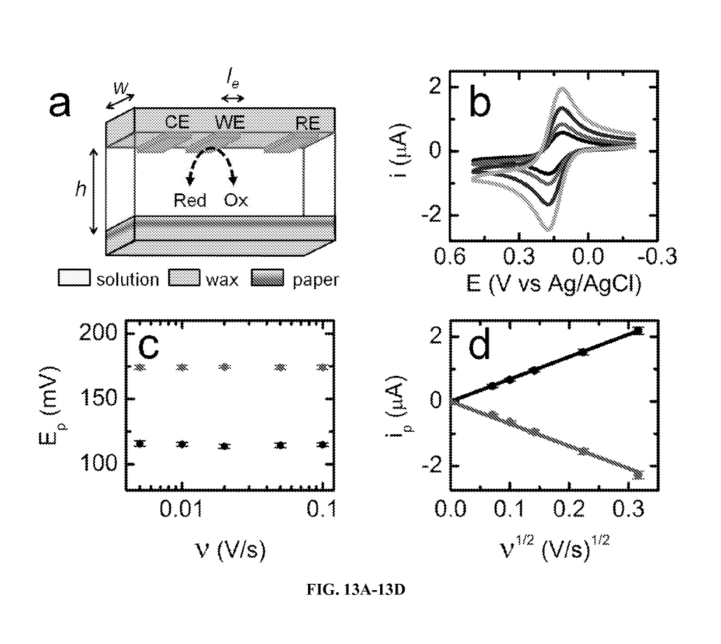

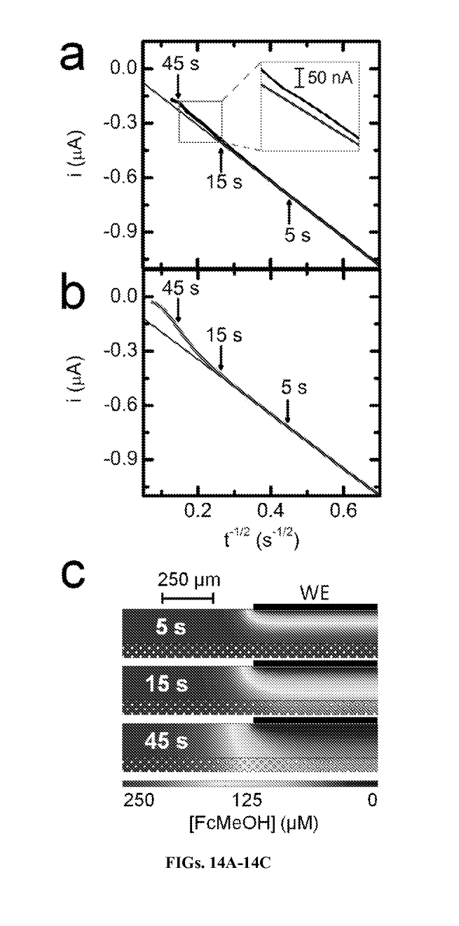

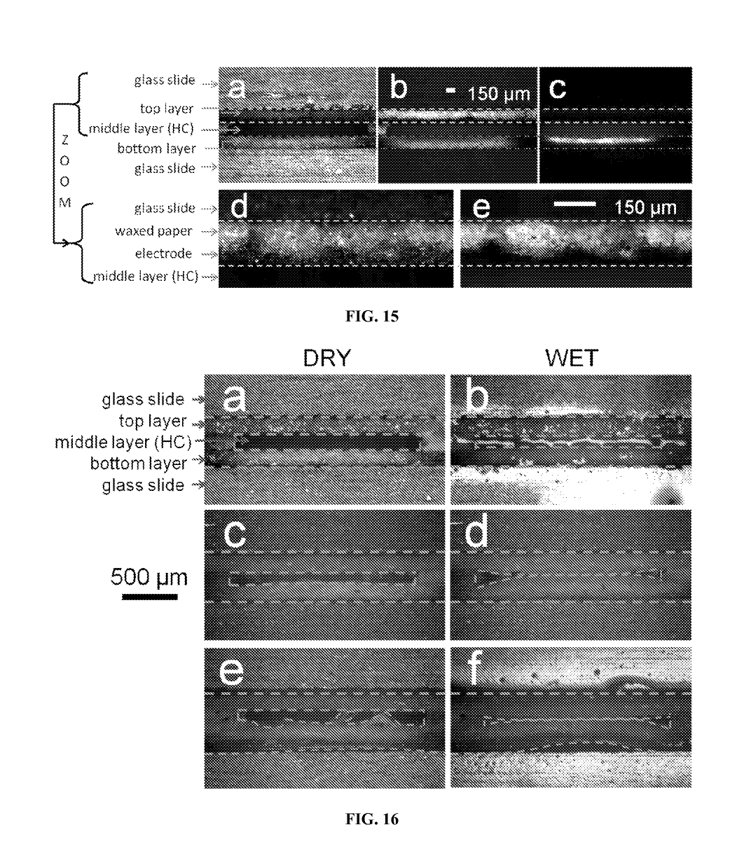

The first part of this study focuses on HC electrochemistry in the absence of convection. The configuration of the HC electrochemical cell is illustrated in FIG. 12A, and cross-sectional micrographs are provided in FIG. 15. The cell consists of three wax patterned paper layers having a thickness of 170±10 μm. A channel cut from the middle paper layer defines the HC, which is 2 mm wide (w), 170 μm high (h), and 30 mm long. The bottom-most layer is partially waxed, so that the bottom of the device is wax but the floor of the channel is unwaxed (and hence hydrophilic and porous) to a depth of 70±10 μm. A complete description of the thickness of each layer is provided in Table 1 below and FIG. 6G.

TABLE 1
Thickness of the layers in the Hollow Channel-PAD.

| | Thickness (μm) | | | | |
|---|---|---|---|---|---|
| | | | | | Paper |
| | Top | Middle | Bottom | Electrode | Floor |
| Laser Cut | 170 ± 10 | 170 ± 10 | 160 ± 10 | 50 ± 10 | 70 ± 10 |
| Razor Blade | 180 ± 10 | 160 ± 10 | 180 ± 10 | — | 80 ± 10 |

Cross-sectional micrographs of dry and wet HCs are compared in FIG. 16. The micrographs show that the dry channels have a nearly perfect rectangular cross section, but that significant structural distortion occurs when the paper is wetted. Accordingly, the measured dimensions of dry HC-PADs only provide an estimate of the HC size and clearly do not reflect the operando dimensions. The working, counter, and reference electrodes, (WE, CE, and RE, respectively) are screen-printed directly on the ceiling of the HC (FIG. 12A). For these experiments, the WE and CE are made with a carbon paste while the reference is made with a Ag/AgCl paste. These electrodes are 2 mm long and span the entire width of the channel.

The electrochemical behavior of the HC-PADs was characterized by cyclic voltammetry (CV) using FcMeOH as a redox probe. These experiments were carried out by flowing a solution containing 250 μM FcMeOH and PBS 1× through the HC for 5 min, stopping the flow, and then recording CVs at scan rates (v) between 10 and 100 mV/s (FIG. 12B). The resulting anodic and cathodic peak potentials (Eₚ) are plotted as a function of v in FIG. 12C, and the anodic and cathodic peak currents (iₙ) are plotted as a function of v^{1/2} in FIG. 12D. The straight lines in FIG. 12D are values of iₚ calculated using the Randles-Sevcik equation. The error bars in both plots correspond to standard deviations for measurements obtained from three independently prepared HC-PADs. The coefficients of variation, defined as the standard deviation divided by the average, are 2% and 10% for Eₚ and iₚ, respectively, indicating good device-to-device reproducibly.

The shape of the CVs in FIG. 12B, the peak separations of 59±3 mV observed in FIG. 12C, and the linear variation of iₚ with v^{1/2} are characteristic of a reversible electrochemical system acting under one-dimensional (1D) semi-infinite diffusion. Because the diffusion coefficients of the reduced and oxidized forms of FcMeOH are nearly the same (Dₒₓ=D_{red}=6.7×10⁻⁶ cm²/s), the formal potential, E°′, is equal to the average of the peak potentials: 145 mV vs Ag/AgCl. This value is close to the literature value of 150 mV vs Ag/AgCl. We also observed that the potential of the screen-printed Ag/AgCl reference electrode is stable for at least 30 min, which is also the approximate lifetime of a HC-PAD. The results in FIG. 12 demonstrate that the non-idealities of the system, which include the roughness and wetability of the wax and paper channels walls, conductivity of the electrodes, and the constrained channel geometry, do not substantially affect the performance or reproducibility of HC-PADs over the range of experimental variables considered here.

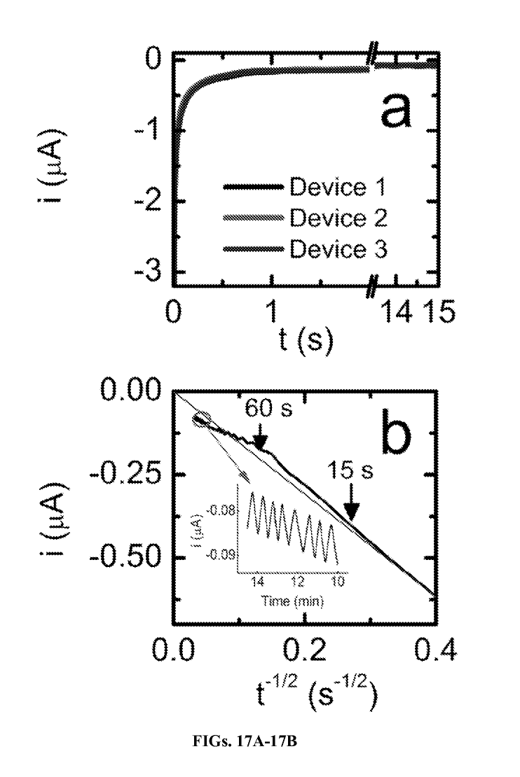

In addition to cyclic voltammetry, chronoamperometry (CA) using the HC-PAD shown schematically in FIG. 12A was also conducted. Current, corresponding to the mass-transfer-limited oxidation of FcMeOH, is plotted as a function of t^{-1/2} in FIG. 14A (black line) for times between 2 and 60 s. CAs measured at longer times are provided in FIG. 17. The blue line in FIG. 14A is a plot of the Cottrell equation for this system. At short times (<15 s), a linear relationship between i and t^{-1/2} was observed for the experimental CA. This relationship is in agreement with the Cottrell equation, which describes the mass transfer-limited current under the 1D semi-infinite boundary condition. After ˜15 s, the magnitude of the current decreases faster than predicted by the Cottrell equation (inset in FIG. 14A), resulting in a noticeable deviation from ideality. The magnitude of the deviation at 45 s is 30±10 nA (measured using three independent devices).

To gain additional insight into the behavior of the CA at t>˜15 s, a numerical simulation of the CA experiment was obtained. For the simulation, the paper floor was modeled as an organized porous layer in which FcMeOH freely diffuses in the pores, but not through the solid cellulose fibers. The red line in FIG. 14B is the simulated CA. The blue line is a linear extrapolation of the portion of the simulated CA between 2 and 7 s. Between 2 and 15 s, the simulated current varies linearly with t^{1/2}. However, after 15 s the magnitude of the simulated current decreases faster than would be expected based on Cottrell behavior (blue line). These observations are in qualitative agreement with the experimental data shown in FIG. 14A. At 45 s the deviation between the simulated CA and the Cottrell (blue) line (˜90 nA) is three times larger than the experimental deviation (30±10 nA). Possible causes could be associated with the low currents and long time scale of the experiments (see FIG. 17).

FIG. 14C shows three concentration profiles, corresponding to the three times (5, 15, and 45 s) indicated in FIG. 14, obtained from the finite element simulation. These snapshots show that at 5 s the diffusion layer thickness is still smaller than the height of the channel, and thus diffusion of FcMeOH can be considered as semi-infinite. At ˜15 s the edge of the diffusion layer (indicated by a light red color) completely penetrates the paper floor (indicated by the white dots). This corresponds to the onset of deviation from 1D semi-infinite diffusion observed in FIG. 14B. After 45 s, the diffusion layer has expanded further into the floor of the HC, significantly depleting the concentration of FcMeOH directly below the electrode (indicated by the thick black line labeled WE). Clearly, the constraint of the diffusion layer by the floor of the channel explains the decrease in current observed in the CAs at t>15 s.

For poly(dimethylsiloxane) (PDMS) microchannels, it has previously been shown that constraint of the diffusion layer can affect the electrochemical response yielding, in extreme cases, a “thin layer” regime. Under the experimental conditions used here the current does not drop to zero as expected for an ideal thin layer electrochemical cell. The primary reason for this observation is that the diffusion layer continues to extend axially along the channel length (FIG. 14C). However, under no-flow conditions most of the volume of the HC below the electrode is probed by diffusion after only 15 to 45 s.

Laminar Flow.

The nature of the flow regime within the channels of the HC-PADs is discussed below. To carry out these experiments, the HC-PAD design illustrated schematically in FIG. 18A was used. This device consists of a “Y” shaped inlet that merges into a single main channel. Toward the center of the main channel, the stream is split again into two separate channels. If the flow is turbulent, then the solutions are expected to quickly mix after they merge at the junction of the “Y”. In contrast, laminar flow leads to slow mixing by diffusion only.

Observation of the interior of the channel through the transparent plastic holder (FIG. 18A) shows that two dye solutions having different colors do not mix while flowing in the main channel. That is, after the two colored solutions are directed into the same main channel and subsequently separated, there is no visual evidence of mixing. This result suggests that fluid flow is laminar.

To confirm and quantify this result in the presence of the top wax layer supporting the electrodes (FIG. 18B), electrochemistry was used to monitor the composition of the solution in the device. In the design shown in FIG. 18B, one WE is placed within each of the two separated streams so that the composition of each can be independently analyzed. For that experiment, the CE (Pt wire) and RE (glass Ag/AgCl, 1 M KCl) were placed in the outlet reservoir. Two 0.5 M NaCl solutions, one containing 1.0 mM FcDM and the other 1.0 mM Fe(phen)3SO4 (E°′=0.268 V vs Ag/AgCl, 1 M KCl and 0.890 V vs Ag/AgCl, 1 M KCl), respectively, as determined by voltammetry) were introduced into the two inlets.

The CVs shown in FIG. 18B were obtained in the two separate branches of the HC after the flow stopped. Only FcDM was detected in the blue channel while mainly Fe(phen)₃SO₄ was observed in the red channel. A trace of FcDM was present in the red channel, which might be because of slightly unequal heights of the solutions at the inlets and hence different fluid velocities. Similar effects have been observed by Osborn et al. in paper devices. These results indicate that the solutions of FcDM and Fe(phen)₃SO₄ do not mix significantly while flowing in the main channel, and therefore flow in HC-PADs is laminar. The experimental observation of laminar flow is further confirmed by the Reynolds number, Re, which is always <5 in the experiments.

Determination of Flow Rate.

To complete the characterization of flow in HC-PADs, the relationship between the flow rate and the pressure drop within the HC was investigated. The pressure drop (P) was controlled by adjusting the height difference (ΔH) between the columns of liquid in the inlet and outlet reservoirs (FIG. 22A). The value of P was calculated using eq 1 (above). Here, ρ is the density of water at 25° C. (997 kg/m³) and g is the gravitational constant. Note that there was some variation in ΔH during the course of each experiment because of liquid transferring from the inlet to the outlet, but this differential was maintained below 10% to ensure a nearly constant flow rate.

The average linear flow rate (uₐᵥ) was measured by electrochemistry using the generation-collection experiment depicted in FIG. 22A. In this experiment, two WEs having a fixed edge-to-edge separation of l_{G-C}=11.5 mm (FIG. 22A), were defined in the HC, while the CE (Pt wire) and RE (SCE) were positioned in the outlet reservoir. The generation-collection experiment is initiated by stepping the potential of the generator electrode from −0.200 V to 0.600 V vs SCE under flowing conditions. This results in oxidation of FcMeOH to FcMeOH⁺. The latter then flows downstream to the collector electrode, which is held at a constant reducing potential of −0.200 V vs SCE to reduce FcMeOH⁺ back to FcMeOH. Typical CAs for the generator and collector electrodes are shown in FIG. 22B. The reduction of the FcMeOH⁺ at the collector electrode gives rise to a sudden increase of cathodic current at t=_{G-C}, indicated by the red arrow in FIG. 22B. After a specified period of time, the currents at the generator and collector electrodes approach limiting values corresponding to i_{L}^{gen} and i_{L}^{col}, respectively. The time delay, t=_{G-C}, between the initial oxidation of FcMeOH at the generator electrode and the initial reduction of FcMeOH⁺ at the collector electrode corresponds to the time necessary for the FcMeOH⁺ to travel the distance l_{G-C}. Values of t_{G-C} were measured for independently fabricated HC-PADs at different pressures (Table 2), and then t_{G-C} was converted into uₐᵥ. The values of uₐᵥ are plotted as a function of pressure in FIG. 22C. As reported previously for plastic microfluidic devices, uₐᵥ varies linearly with pressure. The slope of the best least-squares fit to the experimental data (black line in FIG. 22C) is 2.7±0.2 mm/(s mbar). The coefficient of variation of uₐᵥ within a single device is 11% and from device to device 17%.

TABLE 2
Time delays as a function of pressure.

| | Device 1 | | Device 2 | | Device 3 | |
|---|---|---|---|---|---|---|
| P (mbar) | t_{G-C} (s) | SD (s) | t_{G-C} (s) | SD (s) | t_{G-C} (s) | SD (s) |
| 0.3 | 9.3 | 0.7 | | | | |
| 0.9 | 3.2 | 0.1 | | | | |
| 1.2 | 2.4 | 0.1 | | | | |
| 1.5 | 2.0 | 0.6 | 3.4 | 0.1 | | |
| 2.1 | | | | | 2.3 | 0.1 |
| 2.9 | 1.1 | 0.4 | 1.5 | 0.1 | 1.5 | 0.1 |
| 3.6 | | | | | 1.3 | 0.1 |
| 4.2 | | | 1.1 | 0.1 | 0.9 | 0.1 |
| 4.5 | 0.8 | 0.1 | 0.9 | 0.1 | | |

To compare the flow rate in HC-PADs with traditional PADs, a generation-collection experiment was conducted using a device identical to the HC-PAD, except that the cellulose fibers were left in the channel. The variation of uₐᵥ with P in the paper-channel PAD was found to be 0.0056±0.0002 mm/(s mbar), or 480 times smaller than in HC PADs. This result simply illustrates that pressure-driven flow through a channel obstructed by cellulose fibers is much slower than through a HC.

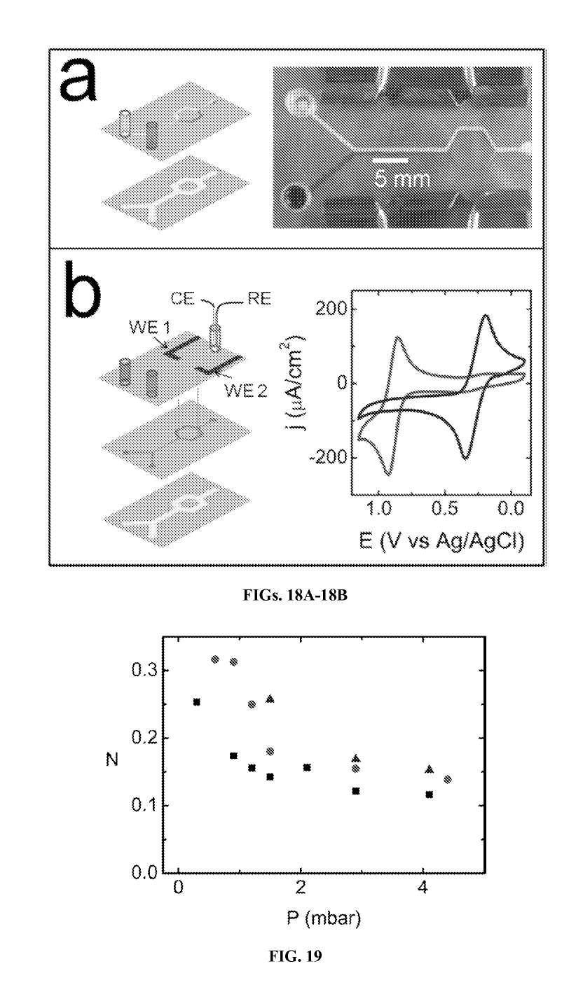

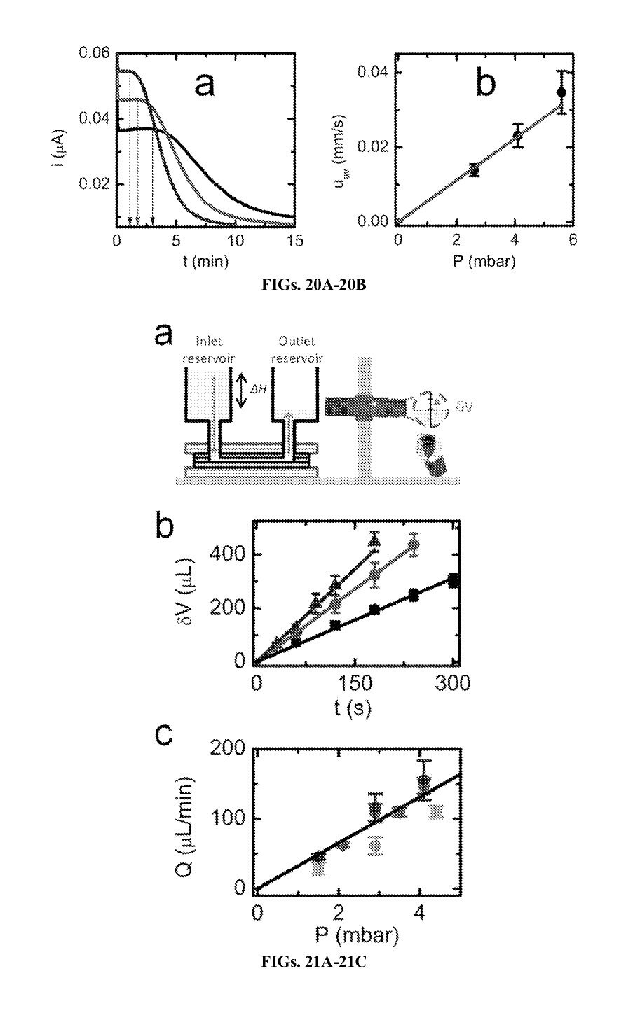

The experimentally determined values of i_{L}^{gen} and i_{L}^{col} (FIG. 22B) were used to calculate the collection efficiency (N) of the HC-PADs. Under experimental conditions, N varies between 0.1 and 0.3 for pressures ranging from 0.3 to 4.5 mbar, respectively (values of N measured for several pressures and devices are provided in FIG. 19). The measured values of N in the HC-PADs are comparable to values observed in glass and plastic microfluidic devices.

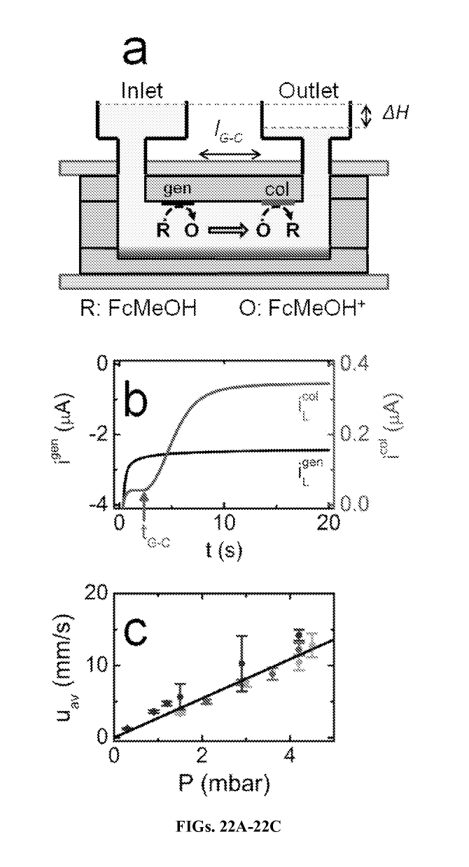

The volumetric flow rate (Q) in HC-PADs was also measured by monitoring the variation of the liquid height in the outlet reservoir as a function of time (FIG. 21). The agreement between the electrochemical generation-collection measurement of uₐᵥ and the optical measurement of Q is qualitative. By comparing the volumetric flow rate and the linear flow rate determined by electrochemistry, the average cross-sectional area of the HC was determined to be 0.19±463 0.03 mm². This value is 44±9% smaller than the value measured using cross-sectional micrographs of dry HCs. The various nonidealities of the paper platform, such as structural deformation, roughness, and degree of hydrophilicity, are likely contributors to this observation.

The volumetric flow rate, Q, was measured using an optical method. A macroscope (Macroscope 8×30, RF Inter-Science Co., NY) was focused on the interface between the liquid, the air, and the wall of the plastic reservoir located at the outlet of the device (FIG. 21A). The view through the macroscope is represented in the blue dashed circle. The variation of liquid height in the outlet reservoir was measured as a function of time using the scale bar integrated into the macroscope and a timer. This value was then converted into a change in volume (δV) using the cross-sectional area of the reservoir (6.79 cm²). The change in volume in the outlet reservoir is plotted in FIG. 21B as a function of time. By adjusting the difference of liquid height (ΔH) between the inlet and outlet reservoir the change of volume can be measured for different pressures (black, red, and blue lines in FIG. 21B). Under the experimental conditions, δV was always observed to vary linearly with time. The straight lines in FIG. 21B are linear fits of the data, and the slope corresponds directly to the value of Q. Values of Q obtained using three independently fabricated devices and several pressures are plotted in FIG. 21C. The coefficient of variation of Q is 12%. The black line in FIG. 21C is a linear fit to all of the measured values of Q. The slope (30±2 μL/(min mbar)) gives the variation of Q as a function of the pressure drop within the HC. The values of uₐᵥ obtained by electrochemistry and Q obtained by optical method are related by the equation below.

Q=A uₐᵥ

where A is the cross-sectional area of the void part of the HC. A comparison of the experimental values of uₐᵥ and Q can thus provide an estimate of the cross section of the device when operating. In this case, the apparent value of A is found to be 0.19±0.03 mm². If the width of the channel is 2.0±0.2 mm, then the channel height, h, is only 95±25 μm; that is, ˜44±15% smaller than the value (h=170 μm) measured by microscopy.

Electrochemistry in the Presence of Convection.

The reproducibility and predictability of flow rates within HCs are ideal for coupling convection to electrochemical detection. In this subsection, the effect of the flow rate on the current is qualitatively and quantitatively analyzed using convection diffusion theories and numerical simulations.

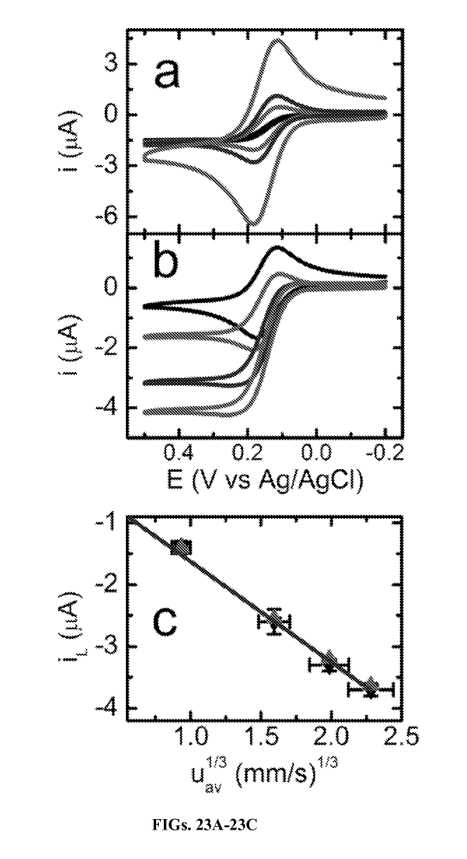

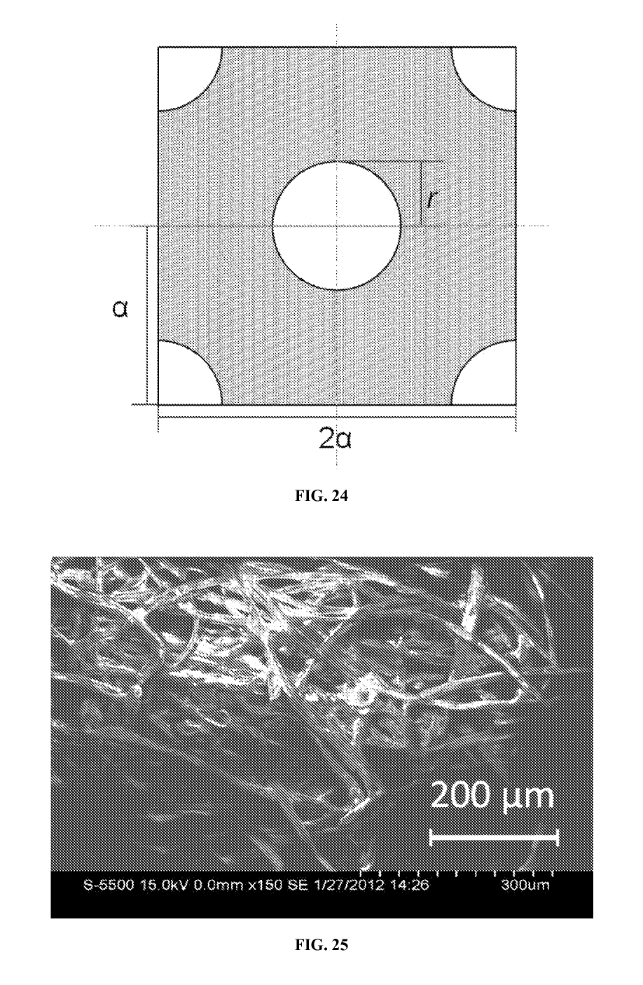

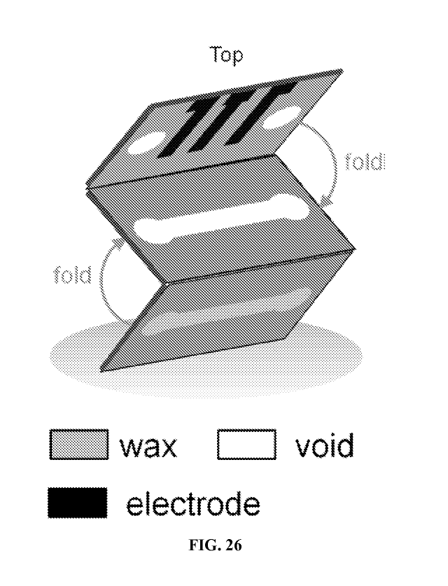

A HC-PAD similar to the one presented in FIG. 12A, that is with the WE (carbon paste), CE (carbon paste) and RE (Ag/AgCl paste) placed directly in the HC, was used to carry out the experiments shown in FIG. 23. In this case, a solution containing 250 μM FcMeOH and PBS 1× is flowed through the device by gravity (as shown in FIG. 22A). FIG. 23A shows CVs recorded as a function of v at a constant pressure of 0.3 mbar. CVs recorded at a constant value of v=50 mV/s and different pressures are plotted in FIG. 23B. When P increases and/or v decreases, the shape of the CVs changes progressively from the shape observed in FIG. 12B (characteristic of 1D semi-infinite diffusion), to a sigmoidal shape (characteristic of steady-state mass transfer).

When convection dominates, the current tends toward a constant, mass-transport-limited value, i_{L}. FIG. 23C shows the value of i_{L}, obtained at several different pressures, as a function of (uₐᵥ)^{1/3} (black triangles). Here the value of uₐᵥ was calculated using the value of P applied at the inlet of the device and the slope of the best-fit line in FIG. 22C. The blue line in FIG. 23C is a linear fit of the experimental data, and the red triangles, which are nearly superimposed on the experimental data, are the limiting currents calculated by numerical simulation. The linear variation of i_{L} with (uₐᵥ)^{1/3} corresponds to the “Levich” mass transfer regime. Under these conditions, convection dominates diffusion and several approximations can be made to obtain an analytical relation between the steady-state limiting current and the linear flow rate. From the slope of the linear fit (FIG. 23C), the apparent height (h) of the channel was calculated to be 148 μm. This value reflects the height of the wetted channel, which as discussed earlier in the context of FIG. 16, is smaller than that of the dry channel (˜170 μm).

The calculated channel height (148 μm) was used with the other experimental parameters to carry out a numerical simulation of convection and diffusion in a HC. The experimentally determined value of uₐᵥ (obtained from the fit in FIG. 22C) and no-slip boundaries were used to solve the Navier-Stokes equation and hence obtain the flow profile in the HC. The concentration of FcMeOH at the electrode was set to zero (that is, the mass-transport-limited condition). A 1.5% difference is observed between the simulation and the experimental data. The numerical simulation indicates that under our experimental conditions a Levich regime is expected, in agreement with the experimental result.

The agreement between the experimental data and the simulation suggests that the approximations invoked for the simulations (the no-slip boundaries and the channel height) are reasonable.

Summary and Conclusions

HC-PADs provide reproducible, quantitative, and predictable electrochemical data. For example, in absence of convection two different regimes are observed: one for short times (<15 s), representing 1D semi-infinite diffusion, and a second case (>15 s), where the diffusion layer extends through the entire height of the channel. In the presence of convection, the electrochemical data are reproducible and quantitatively exhibit Levich behavior.

Fast pressure flow can be initiated using just a drop of fluid, and under the conditions described here flow is laminar and the average linear flow rate varies linearly with P from 0.8 mm/s to 12 mm/s. The flow in HC-PADs and plastic-based devices is similar.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

We claim:

1. A paper-based microfluidic device comprising
a hollow channel fluidly connecting a fluid inlet to a fluid outlet,
wherein the hollow channel comprises a fluid flow path defined by a floor and two or more side walls, and
wherein the floor comprises a hydrophilic material.

2. The device of claim 1, wherein the hollow channel has a height of about 10 μm to about 750 μm.

3. The device of claim 2, wherein the hollow channel has a height of about 25 μm to about 300 μm.

4. The device of any of claims 2-3, wherein the hollow channel has a width of about 0.1 mm to about 50 mm.

5. The device of any of claims 2-4, wherein the hollow channel has a width of about 0.1 mm to about 10 mm.

6. The device of any of claims 1-5, wherein the device comprises a plurality of hollow channels.

7. The device of any of claims 1-6, wherein the side walls of the hollow channel comprise a hydrophobic material.

8. The device of claim 7, wherein the hydrophobic material comprises paper covalently modified to comprise a hydrophobic agent, paper impregnated with a hydrophobic agent, paper coated with a hydrophobic agent, or combinations thereof.

9. The device of claim 8, wherein the hydrophobic agent is selected from the group consisting of curable polymers, natural waxes, synthetic waxes, polymerized photoresists, alkyl ketene dimers, alkenyl succinic anhydrides, hydrophobic halosilanes, rosins, silicones, fluorinated reagents, fluoropolymers, polyolefin emulsions, resin and fatty acids, or combinations thereof.

10. The device of any of claims 1-9, wherein the hydrophilic material comprises paper.

11. The device of any one of claims 1-10, further comprising an assay reagent in fluid contact with the hollow channel.

12. The device of any of claims 1-11, further comprising a detection device configured to analyze a fluid present in the hollow channel.

13. The device of claim 12, wherein the detection device is selected from the group consisting of an image scanner, a fluorometer, a spectrometer, an electroanalytical device, or a combination thereof.

14. The device of any of claims 12-13, further comprising signal processing circuitry or a processor in communication with the detection device that is configured to obtain information about a fluid present in the hollow channel based on the output from the detection device.

15. The device of any of claims 1-14, further comprising an obstacle disposed in the hollow channel to slow or stop the flow of a fluid through the hollow channel.

16. The device of claim 15, wherein the obstacle is selected from the group consisting of a pillar, bead, paper barrier, hydrophobic weir, and combinations thereof.

17. A microfluidic device comprising
a hollow channel fluidly connecting a fluid inlet to a fluid outlet, and
a bulk conductive electrode in electrochemical contact with the hollow channel.

18. The device of claim 17, wherein the hollow channel comprises a height and a width.

19. The device of claim 18, wherein the height of the hollow channel is about 10 μm to about 750 μm.

20. The device of claim 19, wherein the height of the hollow channel is about 25 μm to about 300 μm.

21. The device of any of claims 18-20, wherein the width of the hollow channel is about 0.1 mm to about 50 mm.

22. The device of any of claims 18-21, wherein the width of the hollow channel is about 0.1 mm to about 10 mm.

23. The device of any of claims 17-22, wherein the device comprises a plurality of hollow channels.

24. The device of any of claims 17-23, wherein the hollow channel comprises a fluid flow path defined by a floor and two or more side walls.

25. The device of claim 24, wherein the side walls of the hollow channel comprise a hydrophobic material.

26. The device of claim 25, wherein the hydrophobic material comprises paper covalently modified to comprise a hydrophobic agent, paper impregnated with a hydrophobic agent, paper coated with a hydrophobic agent, or combinations thereof.

27. The device of claim 26, wherein the hydrophobic agent is selected from the group consisting of curable polymers, natural waxes, synthetic waxes, polymerized photoresists, alkyl ketene dimers, alkenyl succinic anhydrides, hydrophobic halosilanes, rosins, silicones, fluorinated reagents, fluoropolymers, polyolefin emulsions, resin and fatty acids, or combinations thereof.

28. The device of any of claims 24-27, wherein the floor comprises a hydrophilic material.

29. The device of claim 28, wherein the hydrophilic material comprises paper.

30. The device of any one of claims 17-29, further comprising an assay reagent in fluid contact with the hollow channel.

31. The device of claim 30, wherein the assay reagent is disposed on the bulk conductive electrode.

32. The device of any of claims 17-31, further comprising a counter electrode, a reference electrode, or combinations thereof in electrochemical contact with the hollow channel.

33. The device of any of claims 17-32, further comprising a power supply and signal processing circuitry or a processor in electrical communication with the bulk conductive electrode.

34. The device of any of claims 17-33, further comprising an obstacle disposed in the hollow channel to slow or stop the flow of a fluid through the hollow channel.

35. The device of claim 34, wherein the obstacle is selected from the group consisting of a pillar, bead, paper barrier, hydrophobic weir, and combinations thereof.

36. The device of any of claims 17-35, wherein the bulk conductive electrode is selected from the group consisting of wire, mesh, fiber, plate, foil, perforated plate, and perforated foil.

37. A paper-based microfluidic device comprising
(a) a sample deposition layer comprising a top surface, a bottom surface, a fluid inlet defining a path for fluid flow from the top surface of the sample deposition layer to the bottom surface of the sample deposition layer, and a fluid outlet defining a path for fluid flow from the bottom surface of the sample deposition layer to the top surface of the sample deposition layer,
(b) a channel layer comprising a top surface, a bottom surface, a hydrophobic boundary defining a hollow channel within the channel layer, and
(c) a base layer comprising a top surface, a bottom surface, a hemichannel comprising a hydrophilic material disposed within the top surface of the base layer
wherein the sample deposition layer, the channel layer, and the base layer are stacked such that the bottom surface of the sample deposition layer is in fluid communication with the top surface of the channel layer, and the bottom surface of the channel layer is in fluid communication with the top surface of the base layer; and
wherein the channel layer and the base layer are aligned such that when the device is assembled, the hollow channel comprises a fluid flow path defined by a floor comprising the hemichannel of the base layer and two or more side walls comprising the hydrophobic boundary of the channel layer; and
wherein the sample deposition layer, the channel layer, and the base layer are aligned so as to form a path for fluid flow from the fluid inlet through the hollow channel to the fluid outlet.

38. The device of claim 37, further comprising a slip layer is disposed between the sample deposition layer and the channel layer.

39. The device of claim 37 or 38, further comprising a slip layer is disposed between the channel layer and the base layer.

40. The device of any of claims 37-39, wherein the sample deposition layer, the channel layer, and the base layer are fabricated from a single piece of paper that is folded to form the device.

41. The device of any of claims 37-40, wherein the hollow channel has a height and a width.

42. The device of claim 41, wherein the height of the hollow channel is about 10 μm to about 750 μm.

43. The device of claim 42, wherein the height of the hollow channel is about 25 μm to about 300 μm.

44. The device of any of claims 41-43, wherein the width of the hollow channel is about 0.1 mm to about 50 mm.

45. The device of any of claims 41-44, wherein the width of the hollow channel is about 0.1 mm to about 10 mm.

46. The device of any of claims 37-45, wherein the device comprises a plurality of hollow channels.

47. The device of any of claims 37-46, wherein the hydrophobic boundary comprises paper covalently modified to comprise a hydrophobic agent, paper impregnated with a hydrophobic agent, paper coated with a hydrophobic agent, or combinations thereof.

48. The device of claim 47, wherein the wherein the hydrophobic agent is selected from the group consisting of curable polymers, natural waxes, synthetic waxes, polymerized photoresists, alkyl ketene dimers, alkenyl succinic anhydrides, hydrophobic halosilanes, rosins, silicones, fluorinated reagents, fluoropolymers, polyolefin emulsions, resin and fatty acids, or combinations thereof.

49. The device of any of claims 37-48, wherein the hydrophilic material comprises paper.

50. The device of any one of claims 37-49, further comprising an assay reagent in fluid contact with the hollow channel.

51. The device of any of claims 37-50, further comprising a detection device configured to analyze a fluid present in the hollow channel.

52. The device of claim 51, wherein the detection device is selected from the group consisting of an image scanner, a fluorometer, a spectrometer, an electroanalytical device, or a combination thereof.

53. The device of any of claims 51-52, further comprising signal processing circuitry or a processor in communication with the detection device that is configured to obtain information about a fluid present in the hollow channel based on the output from the detection device.

54. The device of any of claims 37-53, further comprising an obstacle disposed in the hollow channel to slow or stop the flow of a fluid through the hollow channel.

55. The device of claim 54, wherein the obstacle is selected from the group consisting of a pillar, bead, paper barrier, hydrophobic weir, and combinations thereof.

56. A microfluidic device comprising a hollow channel fluidly connecting a fluid inlet to a fluid outlet, wherein the hollow channel comprises a fluid flow path defined by a floor, two or more side walls, and a ceiling,
wherein at least one of the floor, the two or more side walls, or the ceiling comprises a hydrophilic material.

57. The device of claim 56, wherein only one of the floor, the two or more side walls, or the ceiling comprises the hydrophilic material.

58. The device of claim 56 or 57, wherein the floor comprises the hydrophilic material.

59. The device of any of claims 56-58, wherein the ceiling comprises the hydrophilic material.

60. The device of any of claims 56-59, wherein the hollow channel has a height of about 10 μm to about 750 μm.

61. The device of any of claims 56-60, wherein the hollow channel has a height of about 25 μm to about 300 μm.

62. The device of any of claims 56-61, wherein the hollow channel has a width of about 0.1 mm to about 50 mm.

63. The device of any of claims 56-62, wherein the hollow channel has a width of about 0.1 mm to about 10 mm.

64. The device of any of claims 56-63, wherein the device comprises a plurality of hollow channels.

65. The device of any of claims 56-64, wherein at least one side wall of the hollow channel comprises a hydrophobic material.

66. The device of claim 65, wherein the hydrophobic material comprises paper covalently modified to comprise a hydrophobic agent, paper impregnated with a hydrophobic agent, paper coated with a hydrophobic agent, or combinations thereof.

67. The device of claim 66, wherein the hydrophobic agent is selected from the group consisting of curable polymers, natural waxes, synthetic waxes, polymerized photoresists, alkyl ketene dimers, alkenyl succinic anhydrides, hydrophobic halosilanes, rosins, silicones, fluorinated reagents, fluoropolymers, polyolefin emulsions, resin and fatty acids, or combinations thereof.

68. The device of any of claims 56-67, wherein the hydrophilic material comprises paper.

69. The device of any of claims 56-68, further comprising an assay reagent in fluid contact with the hollow channel.

70. The device of any of claims 56-69, further comprising a detection device configured to analyze a fluid present in the hollow channel.

71. The device of claim 70, wherein the detection device is selected from the group consisting of an image scanner, a fluorometer, a spectrometer, an electroanalytical device, or a combination thereof.

72. The device of any of claims 56-71, further comprising signal processing circuitry or a processor in communication with the detection device that is configured to obtain information about a fluid present in the hollow channel based on the output from the detection device.

73. The device of any of claims 56-72, further comprising an obstacle disposed in the hollow channel to slow or stop the flow of a fluid through the hollow channel.

74. The device of claim 73, wherein the obstacle is selected from the group consisting of a pillar, bead, paper barrier, hydrophobic weir, and combinations thereof.
